(19) United States
(12) Patent Application Publication
(10) Pub. No.: US 2020/0188469 A1
(43) Pub. Date: Jun. 18, 2020

(54) COMPOUNDS AND METHODS FOR INHIBITING CDK5 ALLEVIATE CARDIAC PHENOTYPES IN TIMOTHY SYNDROME AND RELATED CONDITIONS

(71) Applicant: The Trustees of Columbia University in the City of New York, New York, NY (US)

(72) Inventors: Masayuki YAZAWA, New York, NY (US); LouJin SONG, Cambridge, MA (US)

(21) Appl. No.: 16/500,670

(22) PCT Filed: Apr. 3, 2018
(86) PCT No.: PCT/US2018/025831

§ 371 Date: Oct. 3, 2019

Related U.S. Application Data
(60) Provisional application No. 62/481,364, filed on Apr. 4, 2017.

(51) Int. Cl. A61K 38/00; C12N 15/113; A61P 25/00; A61P 9/00; A61K 31/52; A61K 31/454; A61K 31/15; A61K 31/5375

(52) U.S. Cl. CPC A61K 38/005; C12N 15/113; A61P 25/00; A61P 9/00; A61K 48/00; A61K 31/454; A61K 31/15; A61K 31/5375; A61K 31/52

(57) ABSTRACT

The present invention relates to compounds and methods for inhibiting CDK5 or the CDK5 pathway for treating long QT syndrome (LQTS), and in particular Timothy Syndrome (TS). Additionally, the invention relates to small molecule and gene therapy based therapies and combinations for treating Timothy Syndrome (TS), and related channelopathies.

### CROSS-REFERENCE TO RELATED APPLICATIONS

This present application claims priority to U.S. Provisional Patent Application Ser. No. 62/481,364 filed Apr. 4, 2017, which is incorporated herein by reference in its entirety.

### GOVERNMENT SUPPORT

This invention was supported in part with government support under grant numbers R00HL111345 and 5F31HL131087 awarded b y National Institutes of Health. The United States Government may have certain rights in the invention.

### FIELD OF THE INVENTION

The present invention relates to compounds and methods for inhibiting CDK5 or the CDK5 pathway for treating long QT syndrome (LQTS), and in particular Timothy Syndrome (TS). Additionally, the invention relates to small molecule based therapies, or gene therapies and combinations for treating Timothy Syndrome (TS), and related channelopathies.

### BACKGROUND

Despite a substantial reduction in age-adjusted rates of death from cardiovascular causes during the past 40 to 50 years, cardiovascular disease remains the most common cause of natural death in developed countries. Sudden death due to cardiac arrhythmia is estimated to account for approximately 50 percent of all deaths from cardiovascular causes (Huikuri H V, et al. (2001)). In specific, the risk of sudden death due to genetic and drug-induced prolongation of QT interval (long QT syndrome, LQTS) is a major concern for patients, clinicians and pharmaceutical companies. Genetic LQTS has an estimated prevalence of 1 in 7,000 individuals and it results from mutations in at least 13 genes that encode cardiac ion channel genes or other regulatory molecules (Crotti L, et al. (2013); Mahida S, et al. (2013); Venetucci L, et al. (2012)). Manifestations of LQTS during fetal or neonatal life usually indicate a severe form of the disease. Drug-induced LQTS is a side effect of many approved drugs and is a common cause of drug failure in clinical trials (Mahida S, et al. (2013), Paakkari I. (2002)). Despite our knowledge of many of the genes that cause LQTS, the mechanisms that underlie LQTS in humans are incompletely understood. Animal models of human LQTS using rodents have proved to be problematic because the mouse resting heart rate is approximately 10 fold faster than that of humans. Mouse cardiomyocytes have different electrical properties from their human counterparts. Previous experiments using rodent models and clinical trials could not predict potential side effects; for example, cisapride had been approved by US FDA as a gastroprokinetc agent. However, it was withdrawn from US market in 2000 because approximately 80 people died due to its side effect that causes QT prolongation, resulting in lethal arrhythmia and ventricular tachycardia (Paakkari I. (2002)). Therefore, in order to investigate the molecular mechanisms of human cardiac diseases and to identify new therapeutics, it is important to develop human cell culture model systems of cardiac arrhythmias associated with LQTS.

Timothy syndrome (TS, Long QT Syndrome Type 8, LQT8) is an autosomal dominant disorder characterized by multisystem dysfunctions including lethal arrhythmia, congenital heart defects and autism (Splawski I, et al. (2004)). The disease is caused by one gain-of-function mutation in the CACNA1C gene encoding L-type voltage-gated calcium channel Cav1.2, and the mutation usually leads to ineffective channel inactivation. There are currently very few options for therapeutic treatment of patients with TS and none of the currently used drugs are very effective. Therefore, there is a need to develop new effective therapeutics for TS. To date, several attempts have been made to develop new therapeutics for treating TS and related conditions. However, the results have exhibited limitations. For example, Roscovitine has been shown to rescue the cardiac phenotypes of TS cardiomyocytes derived from hiPSCs, indicating that Roscovitine could be a new therapeutic compound for TS (Yazawa M, et al. (2011); Song L, et al. (2015)). However, the dose of Roscovitine used to rescue the phenotypes of TS cardiomyocytes was high, which makes this compound not ideal for clinical application. Thus, new compounds including analogs of Roscovitine that can be used at a lower dose and that have few or no side effects are still needed to rescue the phenotypes of TS cardiomyocytes. Alternative therapeutics (such as gene therapy) that can mimic the effects of these compounds and enhance the inactivation of the Cav1.2 channel with TS mutation are needed as well.

### SUMMARY OF THE INVENTION

The present invention relates to methods for inhibiting CDK5 in a subject in need thereof, comprising administering to the subject an effective amount of CR8, Myoseverin B, PHA-793887, DRF053, or any specific chemical inhibitor for CDK5, any combinations thereof, or a pharmaceutically acceptable salt thereof.

In certain embodiments, the subject exhibits one or more symptoms associated with Timothy Syndrome (TS) or a related channelopathy.

In certain embodiments, one or more symptoms exhibit improvement and comprise any one or combination of improvements selected from the group consisting of increasing the spontaneous beating rate, decreasing the contraction irregularity, enhancing the voltage-dependent inactivation of CaV1.2 channels, rescuing the abnormal action potentials; and alleviating the abnormal calcium transients in affected or diseased cardiomyocytes.

In certain embodiments, the method further comprises increasing sigma-1 receptor activity in a subject in need thereof, and further comprises administering to the subject an effective amount of fluvoxamine or PRE-084, or certain of its derivatives, combinations thereof, or a pharmaceutically acceptable salt thereof.

In additional embodiments, the present invention relates to a method for treating Timothy Syndrome (TS) or related channelopathy in a subject in need thereof comprising inhibiting CDK5 activity in the subject in an amount to alleviate at least one symptom associated with TS or related channelopathy.

In certain embodiments, the method comprises administering an effective amount of CR8, Myoseverin B, PHA-793887, DRF053, or any specific chemical inhibitor for CDK5, any combinations thereof, or a pharmaceutically acceptable salt thereof.

In additional embodiments, the present invention relates to a method for treating or reducing risk of a cardiac arrhythmia in a subject in need thereof, comprising administering to the subject a therapeutically effective amount of CR8, Myoseverin B, PHA-793887, Roscovitine, DRF053, or any specific chemical inhibitor for CDK5, any combinations thereof, or a pharmaceutically acceptable salt thereof.

In additional embodiments, the present invention relates to a method for treating Timothy syndrome or related channelopathy in a subject in need thereof comprising inhibiting CDK5 or CDK5 activator p35 in the subject in an amount to alleviate at least one symptom associated with Timothy syndrome or related channelopathy.

In certain embodiments, the inhibition is by gene therapy or shRNA treatment. In additional embodiments, the inhibitor of CDK5 is selected from the group consisting of proteins, nucleic acids, and combinations thereof. In yet further embodiments, the nucleic acid is selected from the group consisting of antisense oligonucleotide, siRNA, shRNA, and combinations thereof. In additional embodiments, the method further comprises administering to the subject a therapeutically effective amount of CR8, Myoseverin B, PHA-793887, Roscovitine, DRF053, or any specific chemical inhibitor for CDK5, any combinations thereof, or a pharmaceutically acceptable salt thereof.

### BRIEF DESCRIPTION OF THE DRAWINGS

FIGS. 1A-E are summaries and tables of Roscovitine analog and CDK inhibitor tests. FIG. 1A is a schematic illustration of Roscovitine analog and CDK inhibitor tests. FIG. 1B is a summary of the CDK targets of the positive Roscovitine analogs and CDK inhibitors. n.d., CDK targets are not determined yet. FIG. 1C is a summary of Roscovitine analog and CDK inhibitor tests. Eighteen other Roscovitine analogs did not show positive effects. FIG. 1D are representative traces from the Matlab-based analysis of the Timothy syndrome cardiomyocyte contractions before treatment and 2 hours after the treatment of 2 μM CR8.

FIG. 1E are graphs illustrating the analysis of contraction irregularity of Timothy syndrome cardiomyocytes before treatment and 2 hours after the treatment of each positive compound (n=10 for the chemical compounds and n=5 for DMSO control from one Timothy syndrome iPSC line. The irregularity value after treatment was normalized to the corresponding irregularity value before treatment for each sample in each group. *P<0.05, “P<0.01; Student's t-test, paired). Ros, Roscovitine. Myo-B, Myoseverin-B. PHA, PHA-793887.

FIGS. 2A-N are graphs and traces showing that CDK5 inhibition alleviated the phenotypes in Timothy syndrome cardiomyocytes. FIG. 2A shows representative voltage-clamp recordings of Ba²⁺ currents in the Timothy syndrome cardiomyocyte with (+CDK5 DN) and without CDK5 DN expression (−CDK5 DN). “1.0(relative)” means that the data points were normalized to the corresponding peak current value to make the traces. FIG. 2B shows voltage-dependent inactivation percentage quantification in Timothy syndrome cardiomyocytes with (n=19) and without CDK5 DN expression (n=7). FIG. 2C shows current-voltage relationships of the Ba²⁺ currents in Timothy syndrome cardiomyocytes with (squares, n=19) and without CDK5 DN expression (circles, n=7) are statistically not significantly different. FIG. 2D are representative paced (0.2 Hz) action potential recordings in the CDK5 DN lentivirus infected (+CDK5 DN) and uninfected TS cardiomyocyte. FIG. 2E are graphs of action potential duration at 90% of repolarization (APD90) quantification in the control cardiomyocytes (n=10 from three lines) and the TS cardiomyocytes with (n=10 from two lines) and without CDK5 DN expression (n=8 from two lines) (One-way ANOVA with Bonferroni post-hoc). FIG. 2F are representative Ca²⁺ transient traces of paced (0.5 Hz) single TS cardiomyocyte infected with the R-GECO1 lentivirus and the YFP lentivirus or the YFP-CDK5 DN lentivirus. Blue dots indicate electrical pulses (2 ms, bipolar pulse, 4 volts). The expression of CDK5 DN alleviated the abnormal paced Ca²⁺ transients in TS cardiomyocytes. Y-axis, ΔF/F0 for R-GECO1 (calcium fluorescent indicator). FIGS. 2G, H, I, J are graphs showing the analysis of Ca²⁺ transient duration, half decay time, amplitude and integrated calcium transients (area under curve) in the paced TS cardiomyocytes with and without CDK5 DN expression (n=7 for the group without CDK5 DN, n=17 for the group with CDK5 DN). FIG. 2K are representative voltage-clamp recordings of Ba²⁺ currents in single TS cardiomyocyte with CDK5 DN expression before (blue) and after Roscovitine treatment (red, 5 μM, 3 min). FIG. 2L is a graph showing that Roscovitine did not significantly enhance the voltage-dependent inactivation of Cav1.2 in TS cardiomyocytes with CDK5 DN expression (n=4). FIG. 2M are representative recordings of Ba²⁺ currents in the TS cardiomyocyte with (+shRNA) and without (−shRNA) CDK5 shRNA expression. FIG. 2N is a graph showing voltage-dependent inactivation percentage quantification in TS cardiomyocytes with (n=13) and without CDK5 shRNA expression (n=9). The data in FIG. 2C, E, G-J, L and N are mean±s.e.m. All data were from two lines and Student's t-test was used for statistics unless otherwise stated. n.s., not significant; *P<0.05, “P<0.01, ***P<0.005. See Table 1 for the detailed information of the iPSC lines used for each experiment.

FIGS. 3A-I are schematics, blots, and graphs showing direct interaction and phosphorylation between CDK5 and Cav1.2. FIG. 3A is a schematic showing the structure of human Cav1.2/alc subunit. The G406R mutation and five CDK5 consensus sequences in Cav1.2 are shown. FIGS. 3B-C are blots showing co-immunoprecipitation (IP) was performed using FLAG antibody resins with HEK 293T cell lysates expressing YFP-CDK5 and FLAG-Cav1.2 (FIG. 3B), or FLAG-II-III loop (FIG. 3C) or FLAG-carboxyl-terminus (C-term, FIG. 3C). Anti-(α-) human CDK5 and FLAG-tag antibodies were used for immunoblotting (IB).

FIG. 3D is a schematic showing the design of the in vitro kinase assay. The phosphorylation of the substrates by activated CDK5 consumes ATP and produces ADP that is converted into luminescence. FIG. 3E-F are blots showing Wild-type (WT) II-III loop (II-III) and C-terminus (C-term) were phosphorylated by CDK5. PHA-793887 (PHA) and the mutagenesis (II-III Mutant (MT): S783G; C-term 4MT: 51742A/51799A/51882A/T1958A) blocked the phosphorylation n=3 for both PHA groups and n=6 for WT and MT groups. C-term: n=6 for both PHA groups and n=9 for WT and MT groups. **P<0.01; Student's t-test for WT vs MT/4MT; data are mean±s.e.m.). FIG. 3G are graphs showing representative recordings of Ba²⁺ currents in control cardiomyocyte with and without CDK5 WT expression. FIG. 3H are tracings showing CDK5 WT over-expression significantly delayed the voltage-dependent inactivation in control cardiomyocytes (n=14 for −CDK5 WT group and n=12 for +CDK5 WT group from three control lines. *P<0.05; Student's t-test; data are mean±s.e.m.). FIG. 3I are representative calcium transient traces of control cardiomyocytes infected with the R-GECO1 lentivirus and the YFP lentivirus (n=24 from two lines) or the YFP-CDK5 WT lentivirus (n=20 from two lines). Y-axis, ΔF/F0 for R-GECO1 (calcium fluorescent indicator).

FIGS. 4A-E are graphs, blots, and a schematic showing mechanisms underlying the effects of CDK5 inhibition on Timothy syndrome cardiomyocytes. FIGS. 4A-C GAPDH was used to normalize CDK5, CDK5R1 (p35), CDK5R2 (p39) and EGR1 expression in the qPCR analysis (*P<0.05, **P<0.01; Student's t-test; data are mean±s.e.m.). Cardiomyocyte samples from four control lines (Ctrl, n=12 for CDK5, CDK5R1, CDK5R2 and n=9 for EGR1 including two isogenic controls) and two Timothy syndrome lines (TS, n=14 for CDK5, CDK5R1, CDK5R2 and n=9 for EGR1) were tested. FIG. 4D are blots showing that phosphorylated ERK (pERK) and p35 proteins were increased in Timothy syndrome (TS) cardiomyocytes compared with control (Ctrl). FIG. 4E is a schematic presentation of the proposed signaling pathway in Timothy syndrome cardiomyocytes.

### DETAILED DESCRIPTION

Aspects of the present invention relate in part to the molecular mechanism in which CaV1.2 channels are regulated by CDK5. The present data provides new insights into the regulation of cardiac calcium channels and the development of novel therapeutics for Timothy syndrome patients.

In certain embodiments, the present invention relates to methods for inhibiting CDK5 in a subject in need thereof, comprising administering to the subject an effective amount of CR8, Myoseverin B, PHA-793887, DRF053, or any specific chemical inhibitor for CDK5, any combinations thereof, or a pharmaceutically acceptable salt thereof. In certain embodiments, the subject exhibits one or more symptoms associated with Timothy syndrome or a related channelopathy.

In certain embodiments, the present invention relates to methods for treating Timothy syndrome or related channelopathy in a subject in need thereof comprising inhibiting CDK5 in the subject in an amount to alleviate at least one symptom associated with Timothy syndrome or related channelopathy. In certain embodiments, the inhibiting is by gene therapy or shRNA treatment. In certain embodiments, the inhibiting is by administering an effective amount of CR8, Myoseverin B, PHA-793887, DRF053, or any specific chemical inhibitor for CDK5, any combinations thereof, or a pharmaceutically acceptable salt thereof.

In certain embodiments, the present invention relates to methods treating or reducing risk of a cardiac arrhythmia in a subject in need thereof, comprising administering to the subject a therapeutically effective amount of one or more compounds including comprising CR8, Myoseverin B, PHA-793887, DRF053, or any specific chemical inhibitor for CDK5, any combinations thereof, or a pharmaceutically acceptable salt thereof.

Additional aspects include combination treatments using one or more CDK5 inhibitors along with one or more sigma-1 receptor agonists such as fluvoxamine or PRE-084.

### Definitions

Terms have the meanings ascribed to them in the text unless expressly stated to the contrary. It must be noted that, as used herein, the singular forms “a”, “an,” and “the” include plural references unless the context clearly dictates otherwise. In addition, the following terms have the following meanings.

The term “effective amount” of a compound is a quantity sufficient to achieve a desired therapeutic and/or prophylactic effect, for example, an amount which results in the alleviation, prevention of, or a decrease in the symptoms associated with a disease that is being treated, e.g., Long QT syndrome (LQTS), or in particular Timothy Syndrome (TS).

“Activation,” “stimulation,” and “treatment,” as it applies to cells or to receptors, may have the same meaning, e.g., activation, stimulation, or treatment of a cell or receptor with a ligand, unless indicated otherwise by the context or explicitly. “Ligand” encompasses natural and synthetic ligands, e.g., cytokines, cytokine variants, analogues, muteins, and binding compounds derived from antibodies. “Ligand” also encompasses small molecules, e.g., peptide mimetics of cytokines and peptide mimetics of antibodies. “Activation” can refer to cell activation as regulated by internal mechanisms as well as by external or environmental factors. “Response,” e.g., of a cell, tissue, organ, or organism, encompasses a change in biochemical or physiological behavior, e.g., concentration, density, adhesion, or migration within a biological compartment, rate of gene expression, or state of differentiation, where the change is correlated with activation, stimulation, or treatment, or with internal mechanisms such as genetic programming.

“Activity” of a molecule may describe or refer to the binding of the molecule to a ligand or to a receptor, to catalytic activity; to the ability to stimulate gene expression or cell signaling, differentiation, or maturation; to antigenic activity, to the modulation of activities of other molecules, and the like. “Activity” of a molecule may also refer to activity in modulating or maintaining cell-to-cell interactions, e.g., adhesion, or activity in maintaining a structure of a cell, e.g., cell membranes or cytoskeleton. “Activity” can also mean specific activity, e.g., [catalytic activity]/[mg protein], or [immunological activity]/[mg protein], concentration in a biological compartment, or the like. “Activity” may refer to modulation of components of the innate or the adaptive immune systems.

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. “Administration” and “treatment” can refer, e.g., to therapeutic, pharmacokinetic, diagnostic, research, and experimental methods. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell. The term “subject” includes any organism, preferably an animal, more preferably a mammal (e.g., rat, mouse, dog, cat, rabbit) and most preferably a human.

“Treat” or “treating” means to administer a therapeutic agent, such as a composition containing any compound or therapeutic agent of the present invention, internally or externally to a subject or patient having one or more disease symptoms, or being suspected of having a disease or being at elevated at risk of acquiring a disease, for which the agent has therapeutic activity. Typically, the agent is administered in an amount effective to alleviate one or more disease symptoms in the treated subject or population, whether by inducing the regression of or inhibiting the progression of such symptom(s) by any clinically measurable degree. The amount of a therapeutic agent that is effective to alleviate any particular disease symptom (also referred to as the “therapeutically effective amount”) may vary according to factors such as the disease state, age, and weight of the patient, and the ability of the drug to elicit a desired response in the subject. Whether a disease symptom has been alleviated can be assessed by any clinical measurement typically used by physicians or other skilled healthcare providers to assess the severity or progression status of that symptom. While an embodiment of the present invention (e.g., a treatment method or article of manufacture) may not be effective in alleviating the target disease symptom(s) in every subject, it should alleviate the target disease symptom(s) in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

“Treatment,” as it applies to a human, veterinary, or research subject, refers to therapeutic treatment, prophylactic or preventative measures, to research and diagnostic applications. “Treatment” as it applies to a human, veterinary, or research subject, or cell, tissue, or organ, encompasses contact of a CDK5 inhibitor to a human or animal subject, a cell, tissue, physiological compartment, or physiological fluid.

### Pharmaceutical Compositions and Administration

To prepare pharmaceutical or sterile compositions of the present invention, the compound is admixed with a pharmaceutically acceptable carrier or excipient. See, e.g., Remington's Pharmaceutical Sciences and U.S. Pharmacopeia: National Formulary, Mack Publishing Company, Easton, Pa. (1984).

In certain embodiments, an effective amount of the following compound or any specific chemical inhibitor for CDK5, or in combination with gene therapies targeting CDK5 or p35, is administered to a patient in need thereof.

Formulations of therapeutic and diagnostic agents may be prepared by mixing with acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.). Additional agents, such as polysorbate 20 or polysorbate 80, may be added to enhance stability.

Toxicity and therapeutic efficacy of the compositions, administered alone or in combination with another agent, can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index (LD50/ED50). In particular aspects, antibodies exhibiting high therapeutic indices are desirable. The data obtained from these cell culture assays and animal studies can be used in formulating a range of dosage for use in human. The dosage of such compounds lies preferably within a range of circulating concentrations that include the ED50 with little or no toxicity. The dosage may vary within this range depending upon the dosage form employed and the route of administration.

In an embodiment of the invention, a composition of the invention is administered to a subject in accordance with the Physicians' Desk Reference 2003 (Thomson Healthcare; 57th edition (Nov. 1, 2002)).

The mode of administration can vary. Suitable routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal, or intra-arterial.

In particular embodiments, the compound or agents can be administered by an invasive route such as by injection (see above). In further embodiments of the invention, the compound, or pharmaceutical composition thereof, is administered intravenously, subcutaneously, intramuscularly, intraarterially, intra-articularly (e.g. in arthritis joints), or by inhalation, aerosol delivery. Administration by non-invasive routes (e.g., orally; for example, in a pill, capsule or tablet) is also within the scope of the present invention.

In yet another embodiment, the compound such as a CDK5 inhibitor is administered in combination with at least one additional therapeutic agent, such as a sigma-1 receptor agonist or a p35 inhibitor, but not limited to these agents.

“Homology” refers to sequence similarity between two polynucleotide sequences or between two polypeptide sequences when they are optimally aligned. When a position in both of the two compared sequences is occupied by the same base or amino acid monomer subunit, e.g., if a position in each of two DNA molecules is occupied by adenine, then the molecules are homologous at that position. The percent of homology is the number of homologous positions shared by the two sequences divided by the total number of positions compared×100. For example, if 6 of 10 of the positions in two sequences are matched or homologous when the sequences are optimally aligned then the two sequences are 60% homologous. Generally, the comparison is made when two sequences are aligned to give maximum percent homology.

“Isolated nucleic acid molecule” means a DNA or RNA of genomic, mRNA, cDNA, or synthetic origin or some combination thereof which is not associated with all or a portion of a polynucleotide in which the isolated polynucleotide is found in nature, or is linked to a polynucleotide to which it is not linked in nature. For purposes of this disclosure, it should be understood that “a nucleic acid molecule comprising” a particular nucleotide sequence does not encompass intact chromosomes. Isolated nucleic acid molecules “comprising” specified nucleic acid sequences may include, in addition to the specified sequences, coding sequences for up to ten or even up to twenty or more other proteins or portions or fragments thereof, or may include operably linked regulatory sequences that control expression of the coding region of the recited nucleic acid sequences, and/or may include vector sequences.

The phrase “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to use promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that not all progeny will have precisely identical DNA content, due to deliberate or inadvertent mutations. Mutant progeny that have the same function or biological activity as screened for in the originally transformed cell are included. Where distinct designations are intended, it will be clear from the context.

As used herein, “polymerase chain reaction” or “PCR” refers to a procedure or technique in which specific nucleic acid sequences, RNA and/or DNA, are amplified as described in, e.g., U.S. Pat. No. 4,683,195. Generally, sequence information from the ends of the region of interest or beyond is used to design oligonucleotide primers. These primers will be identical or similar in sequence to opposite strands of the template to be amplified. The 5′ terminal nucleotides of the two primers can coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al. (1987) Cold Spring Harbor Symp. Quant. Biol. 51:263; Erlich, ed., (1989) PCR TECHNOLOGY (Stockton Press, N.Y.) As used herein, PCR is considered to be one, but not the only, example of a nucleic acid polymerase reaction method for amplifying a nucleic acid test sample comprising the use of a known nucleic acid as a primer and a nucleic acid polymerase to amplify or generate a specific piece of nucleic acid. As used herein, “germline sequence” refers to a sequence of unrearranged immunoglobulin DNA sequences. Any suitable source of unrearranged immunoglobulin sequences may be used. Human germline sequences may be obtained, for example, from JOINS OLVER® germline databases on the website for the National Institute of Arthritis and Musculoskeletal and Skin Diseases of the United States National Institutes of Health. Mouse germline sequences may be obtained, for example, as described in Giudicelli et al. (2005) Nucleic Acids Res. 33:D256-D261.

“Inhibitors” and “antagonists,” or “activators” and “agonists,” refer to inhibitory or activating molecules, respectively, e.g., for the activation of, e.g., a ligand, receptor, cofactor, a gene, cell, tissue, or organ. A modulator of, e.g., a gene, a receptor, a ligand, or a cell, is a molecule that alters an activity of the gene, receptor, ligand, or cell, where activity can be activated, inhibited, or altered in its regulatory properties. The modulator may act alone, or it may use a cofactor, e.g., a protein, metal ion, or small molecule. Inhibitors are compounds that decrease, block, prevent, delay activation, inactivate, desensitize, or down regulate, e.g., a gene, protein, ligand, receptor, or cell. Activators are compounds that increase, activate, facilitate, enhance activation, sensitize, or up regulate, e.g., a gene, protein, ligand, receptor, or cell. An inhibitor may also be defined as a compound that reduces, blocks, or inactivates a constitutive activity. An “agonist” is a compound that interacts with a target to cause or promote an increase in the activation of the target. An “antagonist” is a compound that opposes the actions of an agonist. An antagonist prevents, reduces, inhibits, or neutralizes the activity of an agonist. An antagonist can also prevent, inhibit, or reduce constitutive activity of a target, e.g., a target receptor, even where there is no identified agonist.

To examine the extent of inhibition, for example, samples or assays comprising a given, e.g., protein, gene, cell, or organism, are treated with a potential activator or inhibitor and are compared to control samples without the inhibitor. Control samples, i.e., samples not treated with antagonist, are assigned a relative activity value of 100%. Inhibition is achieved when the activity value relative to the control is about 90% or less, typically 85% or less, more typically 80% or less, most typically 75% or less, generally 70% or less, more generally 65% or less, most generally 60% or less, typically 55% or less, usually 50% or less, more usually 45% or less, most usually 40% or less, preferably 35% or less, more preferably 30% or less, still more preferably 25% or less, and most preferably less than 25%. Activation is achieved when the activity value relative to the control is about 110%, generally at least 120%, more generally at least 140%, more generally at least 160%, often at least 180%, more often at least 2-fold, most often at least 2.5-fold, usually at least 5-fold, more usually at least 10-fold, preferably at least 20-fold, more preferably at least 40-fold, and most preferably over 40-fold higher.

Endpoints in activation or inhibition can be monitored as follows. Activation, inhibition, and response to treatment, e.g., of a cell, physiological fluid, tissue, organ, and animal or human subject, can be monitored by an endpoint. The endpoint may comprise a predetermined quantity or percentage of, e.g., indicia of inflammation, or cell degranulation or secretion, such as the release of a cytokine, toxic oxygen, or a protease. The endpoint may comprise, e.g., a predetermined quantity of ion flux or transport; cell migration; cell adhesion; cell proliferation; potential for metastasis; cell differentiation; and change in phenotype, e.g., change in expression of gene relating to inflammation, apoptosis, transformation, cell cycle, or metastasis (see, e.g., Knight (2000) Ann. Clin. Lab. Sci. 30:145-158; Hood and Cheresh (2002) Nature Rev. Cancer 2:91-100; Timme, et al. (2003) Curr. Drug Targets 4:251-261; Robbins and Itzkowitz (2002) Med. Clin. North Am. 86:1467-1495; Grady and Markowitz (2002) Annu. Rev. Genomics Hum. Genet. 3:101-128; Bauer, et al. (2001) Glia 36:235-243; Stanimirovic and Satoh (2000) Brain Pathol. 10:113-126).

An endpoint of inhibition is generally 75% of the control or less, preferably 50% of the control or less, more preferably 25% of the control or less, and most preferably 10% of the control or less. Generally, an endpoint of activation is at least 150% the control, preferably at least two times the control, more preferably at least four times the control, and most preferably at least ten times the control.

“Small molecule” is defined as a molecule with a molecular weight that is less than 10 kDa, typically less than 2 kDa, preferably less than 1 kDa, and most preferably less than about 500 Da. Small molecules include, but are not limited to, inorganic molecules, organic molecules, organic molecules containing an inorganic component, molecules comprising a radioactive atom, synthetic molecules, peptide mimetics, and antibody mimetics. As a therapeutic, a small molecule may be more permeable to cells, less susceptible to degradation, and less apt to elicit an immune response than large molecules. Small molecules, such as peptide mimetics of antibodies and cytokines, as well as small molecule toxins, have been described (see, e.g., Casset, et al. (2003) Biochem. Biophys. Res. Commun. 307:198-205; Muyldermans (2001) J. Biotechnol. 74:277-302; Li (2000) Nat. Biotechnol. 18:1251-1256; Apostolopoulos, et al. (2002) Curr. Med. Chem. 9:411-420; Monfardini, et al. (2002) Curr. Pharm. Des. 8:2185-2199; Domingues, et al. (1999) Nat. Struct. Biol. 6:652-656; Sato and Sone (2003) Biochem. J. 371:603-608; U.S. Pat. No. 6,326,482 issued to Stewart, et al).

### Nucleic Acids

The invention also comprises certain constructs and nucleic acids encoding the complete or portions of the CDK5 protein described herein. Certain constructs and sequences, including selected CDK5 inhibitory sequences may be useful in certain embodiments.

Preferably, the nucleic acids hybridize under low, moderate or high stringency conditions. A first nucleic acid molecule is “hybridizable” to a second nucleic acid molecule when a single stranded form of the first nucleic acid molecule can anneal to the second nucleic acid molecule under the appropriate conditions of temperature and solution ionic strength (see Sambrook, et al., supra). The conditions of temperature and ionic strength determine the “stringency” of the hybridization. Typical low stringency hybridization conditions include 55° C., 5×SSC, 0.1% SDS and no formamide; or 30% formamide, 5×SSC, 0.5% SDS at 42° C. Typical moderate stringency hybridization conditions are 40% formamide, with 5× or 6×SSC and 0.1% SDS at 42° C. High stringency hybridization conditions are 50% formamide, 5× or 6×SSC at 42° C. or, optionally, at a higher temperature (e.g., 57° C., 59° C., 60° C., 62° C., 63° C., 65° C. or 68° C.). In general, SSC is 0.15M NaCl and 0.015M Na-citrate. Hybridization requires that the two nucleic acids contain complementary sequences, although, depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the higher the stringency under which the nucleic acids may hybridize. For hybrids of greater than 100 nucleotides in length, equations for calculating the melting temperature have been derived (see Sambrook, et al., supra, 9.50-9.51). For hybridization with shorter nucleic acids, e.g., oligonucleotides, the position of mismatches becomes more important, and the length of the oligonucleotide determines its specificity (see Sambrook, et al., supra, 11.7-11.8).

Inhibitory Nucleic Acids that Hybridize to CDK5

Any number of means for inhibiting CDK5 activity or gene expression can be used in the methods of the invention. For example, a nucleic acid molecule complementary to at least a portion of a human CDK5 encoding nucleic acid can be used to inhibit CDK5 gene expression. Means for inhibiting gene expression using short RNA molecules, for example, are known. Among these are short interfering RNA (siRNA), small temporal RNAs (stRNAs), and micro-RNAs (miRNAs). Short interfering RNAs silence genes through an mRNA degradation pathway, while stRNAs and miRNAs are approximately 21 or 22 nt RNAs that are processed from endogenously encoded hairpin-structured precursors, and function to silence genes via translational repression. See, e.g., McManus et al., RNA, 8(6):842-50 (2002); Morris et al., Science, 305(5688):1289-92 (2004); He and Hannon, Nat Rev Genet. 5(7):522-31 (2004).

“RNA interference, or RNAi” a form of post-transcriptional gene silencing (“PTGS”), describes effects that result from the introduction of double-stranded RNA into cells (reviewed in Fire, A. Trends Genet 15:358-363 (1999); Sharp, P. Genes Dev 13:139-141 (1999); Hunter, C. Curr Biol 9:R440-R442 (1999); Baulcombe. D. Curr Biol 9:R599-R601 (1999); Vaucheret et al. Plant J 16: 651-659 (1998)). RNA interference, commonly referred to as RNAi, offers a way of specifically inactivating a cloned gene, and is a powerful tool for investigating gene function.

The active agent in RNAi is a long double-stranded (antiparallel duplex) RNA, with one of the strands corresponding or complementary to the RNA which is to be inhibited. The inhibited RNA is the target RNA. The long double stranded RNA is chopped into smaller duplexes of approximately 20 to 25 nucleotide pairs, after which the mechanism by which the smaller RNAs inhibit expression of the target is largely unknown at this time. While RNAi was shown initially to work well in lower eukaryotes, for mammalian cells, it was thought that RNAi might be suitable only for studies on the oocyte and the preimplantation embryo.

More recently, it was shown that RNAi would work in human cells if the RNA strands were provided as pre-sized duplexes of about 19 nucleotide pairs, and RNAi worked particularly well with small unpaired 3′ extensions on the end of each strand (Elbashir et al. Nature 411: 494-498 (2001)). In this report, “short interfering RNA” (siRNA, also referred to as small interfering RNA) were applied to cultured cells by transfection in oligofectamine micelles. These RNA duplexes were too short to elicit sequence-nonspecific responses like apoptosis, yet they efficiently initiated RNAi. Many laboratories then tested the use of siRNA to knock out target genes in mammalian cells. The results demonstrated that siRNA works quite well in most instances.

For purposes of reducing the activity of CDK5, siRNAs to the gene encoding the CDK5 can be specifically designed using computer programs. Illustrative nucleotide sequences encoding the amino acid sequences of the various CDK5 isoforms are known and published, e.g., in NCBI Gene No. NP_001157882.1 and NP_004926.1. Furthermore, exemplary nucleotide sequences encoding the amino acid sequences of the various CDK5 isoforms are known and published, e.g., in NCBI Gene No. NM_001164410.2 and NM_004935.3.

Software programs for predicting siRNA sequences to inhibit the expression of a target protein are commercially available and find use. One program, siDESIGN from Dharmacon, Inc. (Lafayette, Colo.), permits predicting siRNAs for any nucleic acid sequence, and is available on the internet at dharmacon.com. Programs for designing siRNAs are also available from others, including Genscript (available on the internet at genscript.com/ssl-bin/app/rnai) and, to academic and non-profit researchers, from the Whitehead Institute for Biomedical Research found on the worldwide web at “jura.wi.mit.edu/pubint/http://iona.wi.mit.edu/siRNAext/.”

Any suitable viral knockdown system could be utilized for decreasing CDK5 mRNA levels—including AAV, lentiviral vectors, or other suitable vectors that are capable of being targeted specifically to the liver. (See Zuckerman and Davis 2015).

Additionally, specifically targeted delivery of shcdk5 mRNA or other CDK5 blocking molecule (nucleic acid, peptide, or small molecule) could be delivered by targeted liposome, nanoparticle or other suitable means.

As described herein we provide methods as well as one or more agents/compounds that silence or inhibit CDK5 for the treatment, prophylaxis or alleviation of TS or related channelopathies, or predisposition to such a condition.

An approach for therapy of such disorders is to express anti-sense constructs directed against CDK5 polynucleotides as described herein, and specifically administering them to cardiomyocytes or other appropriate cells, to inhibit gene function and prevent one or more of the symptoms and processes associated with TS or related channelopathies. Such treatment may also be useful in treating patients who already exhibit TS or related channelopathies. In certain instances, administering at least one additional therapeutic agent may be desired, such as one or more sigma-1 receptor agonists.

Anti-sense constructs may be used to inhibit gene function to prevent TS or related channelopathies. Antisense constructs, i.e., nucleic acid, such as RNA, constructs complementary to the sense nucleic acid or mRNA, are described in detail in U.S. Pat. No. 6,100,090 (Monia et al.), and Neckers et al., 1992, Crit Rev Oncog 3(1-2):175-231.

RNA interference (RNAi) is a method of post transcriptional gene silencing (PTGS) induced by the direct introduction of double-stranded RNA (dsRNA) and has emerged as a useful tool to knock out expression of specific genes in a variety of organisms. RNAi is described by Fire et al., Nature 391:806-811 (1998). Other methods of PTGS are known and include, for example, introduction of a transgene or virus. Generally, in PTGS, the transcript of the silenced gene is synthesised but does not accumulate because it is rapidly degraded. Methods for PTGS, including RNAi are described, for example, in the Ambion.com world wide web site, in the directory “/hottopics/”, in the “mai” file.

Suitable methods for RNAi in vitro are described herein. One such method involves the introduction of siRNA (small interfering RNA). Current models indicate that these 21-23 nucleotide dsRNAs can induce PTGS. Methods for designing effective siRNAs are described, for example, in the Ambion web site described above. RNA precursors such as Short Hairpin RNAs (shRNAs) can also be encoded by all or a part of the cdk5 nucleic acid sequence.

Alternatively, double-stranded (ds) RNA is a powerful way of interfering with gene expression in a range of organisms that has recently been shown to be successful in mammals (Wianny and Zernicka-Goetz, 2000, Nat Cell Biol 2:70-75). Double stranded RNA corresponding to the sequence of a cdk5 polynucleotide can be introduced into or expressed in oocytes and cells of a candidate organism to interfere with CDK5 activity.

CDK5 gene expression may also be modulated by introducing peptides or small molecules which inhibit gene expression or functional activity. Thus, compounds identified by the assays described herein as binding to or modulating, such as down-regulating, the amount, activity or expression of CDK5 polypeptide may be administered to liver hepatocyte cells to prevent the function of CDK5 polypeptide. Such a compound may be administered along with a pharmaceutically acceptable carrier in an amount effective to down-regulate expression or activity CDK5, or by activating or down-regulating a second signal which controls CDK5 expression, activity or amount, and thereby alleviating the abnormal condition.

Alternatively, gene therapy may be employed to control the endogenous production of CDK5 by the relevant cells such as cardiomyoctyes cells in the subject. For example, a polynucleotide encoding a cdk5 siRNA or a portion of this may be engineered for expression in a replication defective retroviral vector, as discussed below. The retroviral expression construct may then be isolated and introduced into a packaging cell transduced with a retroviral plasmid vector containing RNA encoding an anti-cdk5 siRNA such that the packaging cell now produces infectious viral particles containing the sequence of interest. These producer cells may be administered to a subject for engineering cells in vivo and regulating expression of the CDK5 polypeptide in vivo. For overview of gene therapy, see Chapter 20, Gene Therapy and other Molecular Genetic-based Therapeutic Approaches, (and references cited therein) in Human Molecular Genetics, T Strachan and A P Read, BIOS Scientific Publishers Ltd (1996).

In some embodiments, the level of CDK5 is decreased in a cardiomyocyte. Furthermore, in such embodiments, treatment may be targeted to, or specific to, cardiomyocyte cells. The expression of CDK5 may be specifically decreased only in diseased cardiomyocyte cells (i.e., those cells which are predisposed to the heart condition, or exhibiting cardiomyoctye disease already), and not substantially in other non-diseased cardiac cells. In these methods, expression of CDK5 may not be substantially reduced in other cells, i.e., cells which are not cardiomyocyte cells. Thus, in such embodiments, the level of CDK5 remains substantially the same or similar in non-cardiomyocyte cells in the course of or following treatment.

Cardiomyocyte cell specific reduction of CDK5 levels may be achieved by targeted administration, i.e., applying the treatment only to the cardiomyocyte cells and not other cells. However, in other embodiments, down-regulation of CDK5 expression in cardiomyocyte cells (and not substantially in other cell or tissue types) is employed. Such methods may advantageously make use of liver specific expression vectors, for cardiomyocyte expression of for example siRNAs, as described in further detail below.

By “down-regulation” included is any negative effect on the condition being studied; this may be total or partial. Thus, where binding is being detected, candidate antagonists are capable of reducing, ameliorating, or abolishing the binding between two entities. The down-regulation of binding (or any other activity) achieved by the candidate molecule may be at least 10%, such as at least 20%, at least 30%, at least 40%, at least 50%, at least 60%, at least 70%, at least 80%, at least 90% or more compared to binding (or which-ever activity) in the absence of the candidate molecule. Thus, a candidate molecule suitable for use as an antagonist is one which is capable of reducing by at least 10% the binding or other activity.

The term “compound” refers to a chemical compound (naturally occurring or synthesized), such as a biological macromolecule (e.g., nucleic acid, protein, non-peptide, or organic molecule), or an extract made from biological materials such as bacteria, plants, fungi, or animal (particularly mammalian) cells or tissues, or even an inorganic element or molecule. The compound may be an antibody.

Examples of potential antagonists of CDK5 include antibodies, small molecules, nucleotides and their analogues, including purines and purine analogues, oligonucleotides or proteins which are closely related to a binding partner of CDK5, e.g., a fragment of the binding partner, or small molecules which bind to the CDK5 polypeptide but do not elicit a response, so that the activity of the polypeptide is prevented, etc.

In some embodiments, the anti-CDK5 agent is provided as an injectable or intravenenous composition and administered accordingly. The dosage of the anti-CDK5 agent inhibitor may be between about 5 mg/kg/2 weeks to about 10 mg/kg/2 weeks. The anti-CDK5 agent inhibitor may be provided in a dosage of between 10-300 mg/day, such as at least 30 mg/day, less than 200 mg/day or between 30 mg/day to 200 mg/day.

The anti-CDK5 agent may downregulate CDK5 by RNA interference, such as by comprising a Small Interfering RNA (siRNA) or Short Hairpin RNA (shRNA).

CDK5 polypeptides or polypeptide fragments comprising amino acid sequences that are at least about 70% identical, preferably at least about 80% identical, more preferably at least about 90% identical and most preferably at least about 95% identical (e.g., 95%, 96%, 97%, 98%, 99%, 100%) to the mouse CDK5 or human CDK5 amino acid sequences with reference to sequences described above, are contemplated with respect to inhibiting CDK5 expression and or function, when the comparison is performed by a BLAST algorithm wherein the parameters of the algorithm are selected to give the largest match between the respective sequences over the entire length of the respective reference sequences.

Polypeptides comprising amino acid sequences that are at least about 70% similar, preferably at least about 80% similar, more preferably at least about 90% similar and most preferably at least about 95% similar (e.g., 95%, 96%, 97%, 98%, 99%, 100%) to any of the reference CDK5 amino acid sequences when the comparison is performed with a BLAST algorithm wherein the parameters of the algorithm are selected to give the largest match between the respective sequences over the entire length of the respective reference sequences, are also included in constructs and methods of the present invention.

Sequence identity refers to the degree to which the amino acids of two polypeptides are the same at equivalent positions when the two sequences are optimally aligned. Sequence similarity includes identical residues and nonidentical, biochemically related amino acids. Biochemically related amino acids that share similar properties and may be interchangeable are discussed above.

“Homology” refers to sequence similarity between two polynucleotide sequences or between two polypeptide sequences when they are optimally aligned. When a position in both of the two compared sequences is occupied by the same base or amino acid monomer subunit, e.g., if a position in each of two DNA molecules is occupied by adenine, then the molecules are homologous at that position. The percent of homology is the number of homologous positions shared by the two sequences divided by the total number of positions compared×100. For example, if 6 of 10 of the positions in two sequences are matched or homologous when the sequences are optimally aligned then the two sequences are 60% homologous. Generally, the comparison is made when two sequences are aligned to give maximum percent homology.

The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S.F. “Evaluating the statistical significance of multiple distinct local alignments.” in Theoretical and Computational Methods in Genome Research (S. Suhai, ed.), (1997) pp. 1-14, Plenum, N.Y.

In certain aspects, the present invention also provides expression vectors comprising various nucleic acids, wherein the nucleic acid is operably linked to control sequences that are recognized by a host cell when the host cell is transfected with the vector.

### Pharmaceutical Compositions and Administration

To prepare pharmaceutical or sterile compositions of the compositions of the present invention, the viral vectors, inhibitors, or similar compositions may be admixed with a pharmaceutically acceptable carrier or excipient. See, e.g., Remington's Pharmaceutical Sciences and U.S. Pharmacopeia: National Formulary, Mack Publishing Company, Easton, Pa. (1984).

Formulations of therapeutic and diagnostic agents may be prepared by mixing with acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

Toxicity and therapeutic efficacy of the therapeutic compositions, administered alone or in combination with another agent, can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index (LD₅₀/ED₅₀). In particular aspects, therapeutic compositions exhibiting high therapeutic indices are desirable. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration.

In an embodiment of the invention, a composition of the invention is administered to a subject in accordance with the Physicians' Desk Reference 2003 (Thomson Healthcare; 57th edition (Nov. 1, 2002)).

The mode of administration can vary. Suitable routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal, or intra-arterial.

In particular embodiments, the composition or therapeutic can be administered by an invasive route such as by injection (see above). In further embodiments of the invention, the composition, therapeutic, or pharmaceutical composition thereof, is administered intravenously, subcutaneously, intramuscularly, intraarterially, intra-articularly (e.g. in arthritis joints), intratumorally, or by inhalation, aerosol delivery. Administration by non-invasive routes (e.g., orally; for example, in a pill, capsule or tablet) is also within the scope of the present invention.

Compositions can be administered with medical devices known in the art. For example, a pharmaceutical composition of the invention can be administered by injection with a hypodermic needle, including, e.g., a prefilled syringe or autoinjector.

The pharmaceutical compositions of the invention may also be administered with a needleless hypodermic injection device; such as the devices disclosed in U.S. Pat. No. 6,620,135; 6,096,002; 5,399,163; 5,383,851; 5,312,335; 5,064,413; 4,941,880; 4,790,824 or 4,596,556.

Alternately, one may administer the viral vectors, RNAi, shRNA or other CDK5 inhibitors, or related compound in a local rather than systemic manner, for example, via injection of directly into the desired target site, often in a depot or sustained release formulation. Furthermore, one may administer the composition in a targeted drug delivery system, for example, in a liposome coated with a tissue-specific antibody, targeting, for example, the heart, and more specifically cardiomyocytes. The liposomes will be targeted to and taken up selectively by the desired tissue. A summary of various delivery methods and techniques of siRNA administration in ongoing clinical trials is provided in Zuckerman and Davis 2015; Nature Rev. Drug Discovery, Vol. 14: 843-856, December 2015.

The administration regimen depends on several factors, including the serum or tissue turnover rate of the therapeutic composition, the level of symptoms, and the accessibility of the target cells in the biological matrix. Preferably, the administration regimen delivers sufficient therapeutic composition to effect improvement in the target disease state, while simultaneously minimizing undesired side effects. Accordingly, the amount of biologic delivered depends in part on the particular therapeutic composition and the severity of the condition being treated.

Determination of the appropriate dose is made by the clinician, e.g., using parameters or factors known or suspected in the art to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. In general, it is desirable that a biologic that will be used is derived from the same species as the animal targeted for treatment, thereby minimizing any immune response to the reagent.

As used herein, “inhibit” or “treat” or “treatment” includes a postponement of development of the symptoms associated with a disorder and/or a reduction in the severity of the symptoms of such disorder. The terms further include ameliorating existing uncontrolled or unwanted symptoms, preventing additional symptoms, and ameliorating or preventing the underlying causes of such symptoms. Thus, the terms denote that a beneficial result has been conferred on a vertebrate subject with a disorder, disease or symptom, or with the potential to develop such a disorder, disease or symptom.

As used herein, the terms “therapeutically effective amount”, “therapeutically effective dose” and “effective amount” refer to an amount of a viral vector, RNAi, shRNA or other CDK5 inhibitors or inhibitor compound of the invention that, when administered alone or in combination with an additional therapeutic agent to a cell, tissue, or subject, is effective to cause a measurable improvement in one or more symptoms of a disease or condition or the progression of such disease or condition. A therapeutically effective dose further refers to that amount of the compound sufficient to result in at least partial amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously. An effective amount of a therapeutic will result in an improvement of a diagnostic measure or parameter by at least 10%; usually by at least 20%; preferably at least about 30%; more preferably at least 40%, and most preferably by at least 50%. An effective amount can also result in an improvement in a subjective measure in cases where subjective measures are used to assess disease severity.

### Kits

The present invention also provides kits comprising the components of the combinations of the invention in kit form. A kit of the present invention includes one or more components including, but not limited to, the viral vectors, RNAi, shRNA or other CDK5 inhibitors, or CDK5 inhibitor compounds, as discussed herein, in association with one or more additional components including, but not limited to a pharmaceutically acceptable carrier and/or a chemotherapeutic agent, as discussed herein. The viral vectors, RNAi, shRNA or other CDK5 inhibitors, or CDK5-based inhibitor compounds, composition and/or the therapeutic agent can be formulated as a pure composition or in combination with a pharmaceutically acceptable carrier, in a pharmaceutical composition.

In one embodiment, a kit includes the viral vectors, RNAi, shRNA, or other CDK5 inhibitors, or CDK5-based inhibitor compounds/composition of the invention or a pharmaceutical composition thereof in one container (e.g., in a sterile glass or plastic vial) and a pharmaceutical composition thereof and/or a chemotherapeutic agent in another container (e.g., in a sterile glass or plastic vial).

In another embodiment of the invention, the kit comprises a combination of the invention, including the viral vectors, RNAi, shRNA or other CDK5 inhibitors, or CDK5-based inhibitor compounds, along with a pharmaceutically acceptable carrier, optionally in combination with one or more therapeutic agent components formulated together, optionally, in a pharmaceutical composition, in a single, common container.

If the kit includes a pharmaceutical composition for parenteral administration to a subject, the kit can include a device for performing such administration. For example, the kit can include one or more hypodermic needles or other injection devices as discussed above.

The kit can include a package insert including information concerning the pharmaceutical compositions and dosage forms in the kit. Generally, such information aids patients and physicians in using the enclosed pharmaceutical compositions and dosage forms effectively and safely. For example, the following information regarding a combination of the invention may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and patent information.

### General Methods

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif.). Standard methods also appear in Ausbel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra). Standard techniques for characterizing ligand/receptor interactions are available (see, e.g., Coligan, et al. (2001) Current Protocols in Immunology, Vol. 4, John Wiley, Inc., New York).

### EXAMPLES

1. Roscovitine Analog and CDK Inhibitor Tests

A test system and controls were developed using Timothy syndrome iPSCs and isogenic control iPSCs that were generated from the Timothy syndrome iPSCs using Transcription activator-like effector nuclease (TALEN) technology. The isogenic control iPSCs demonstrated a normal karyotype and pluripotency, and the cardiomyocytes derived from the isogenic control iPSCs showed regular calcium transients in calcium imaging and normal voltage-dependent inactivation percentage values in voltage clamp recordings, which are comparable to the cardiomyocytes derived from regular non-isogenic control iPSCs. Thus, these serve as a good model system for identifying new Roscovitine analogs, as described below.

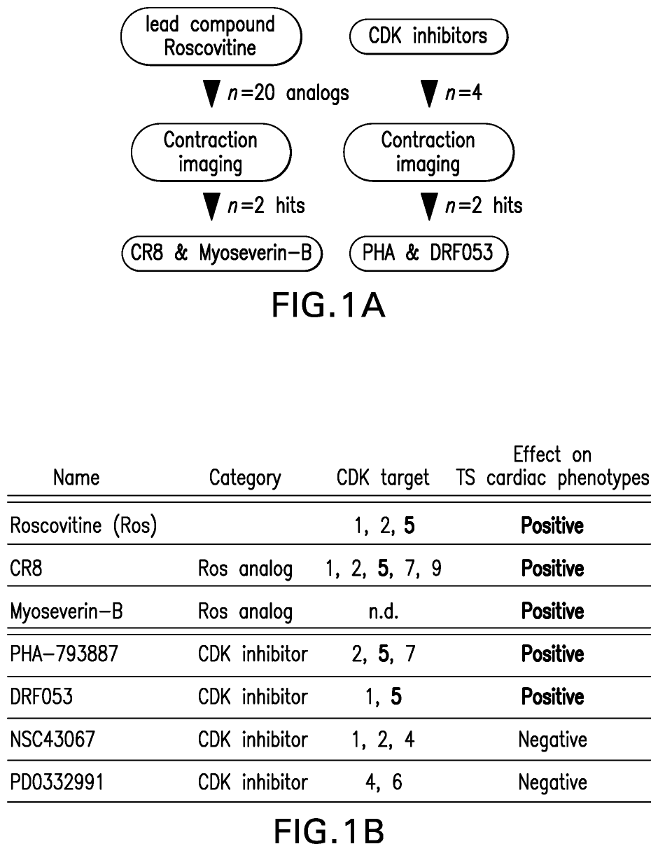

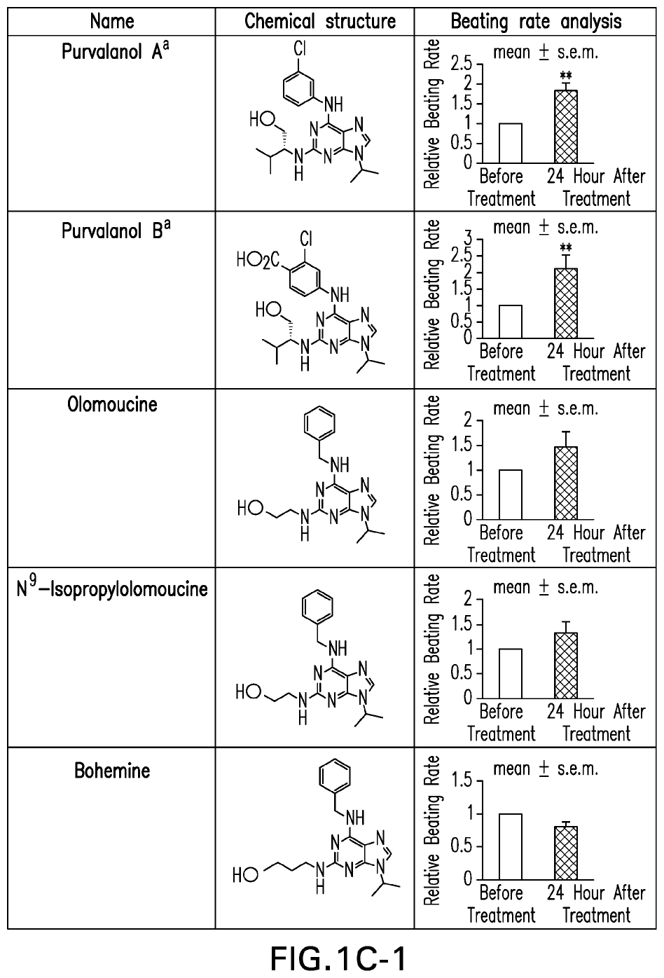

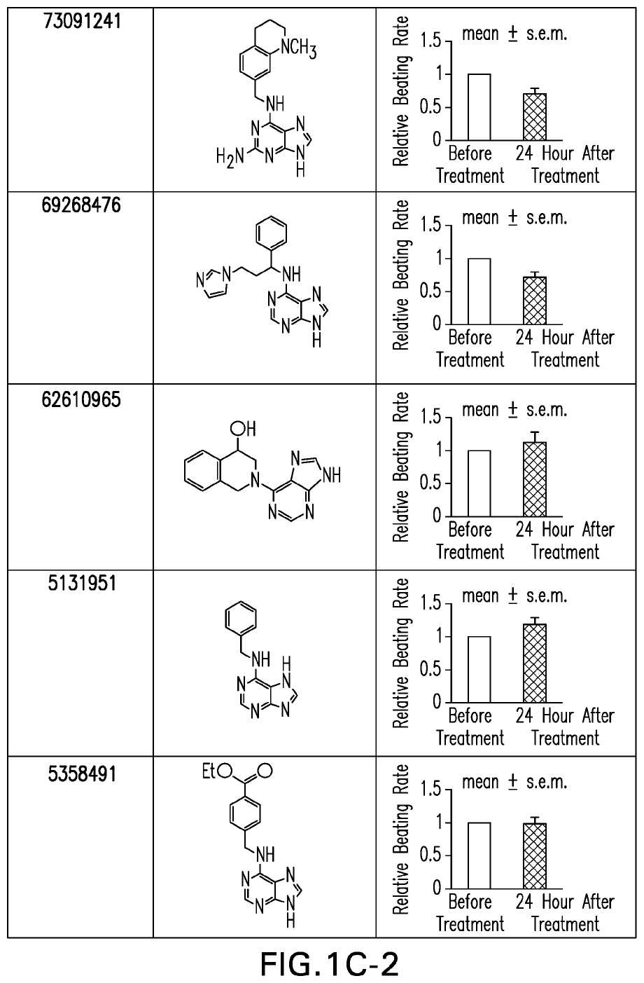

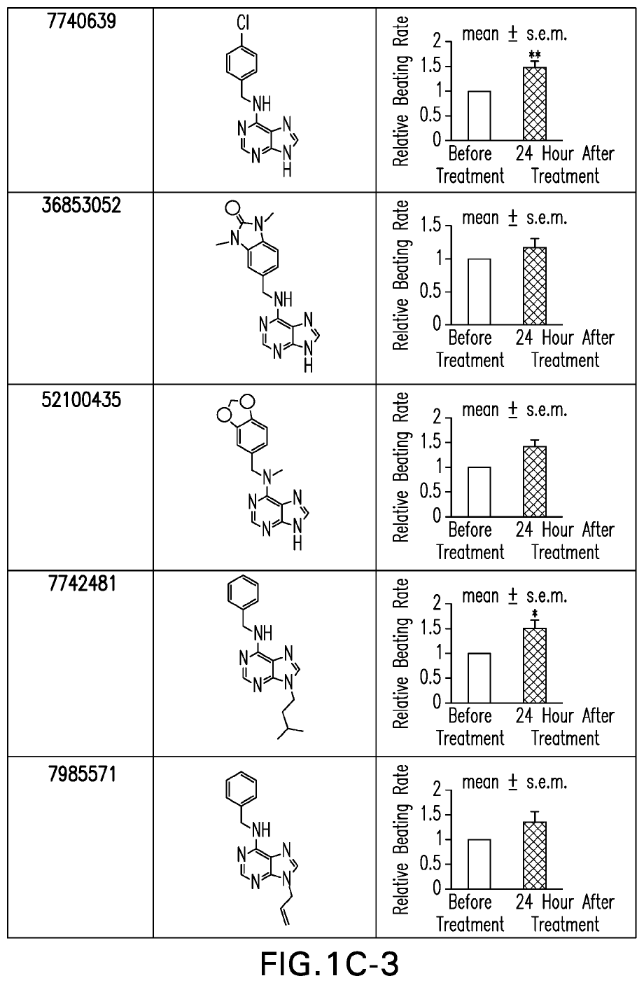

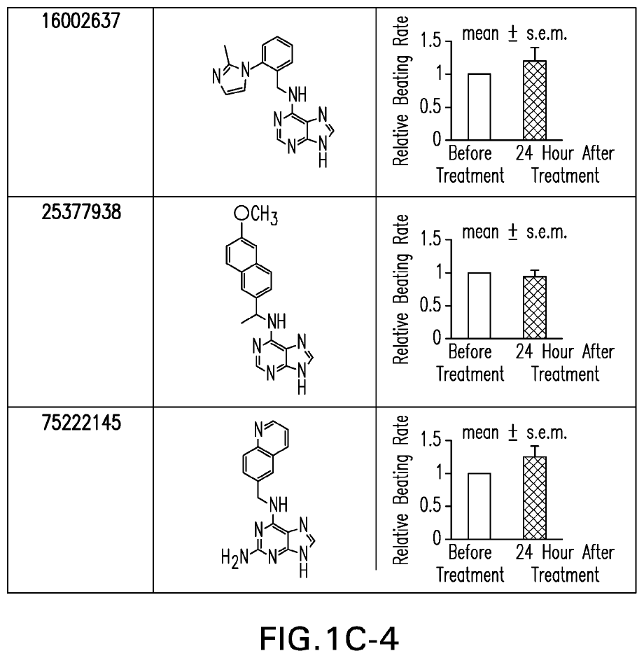

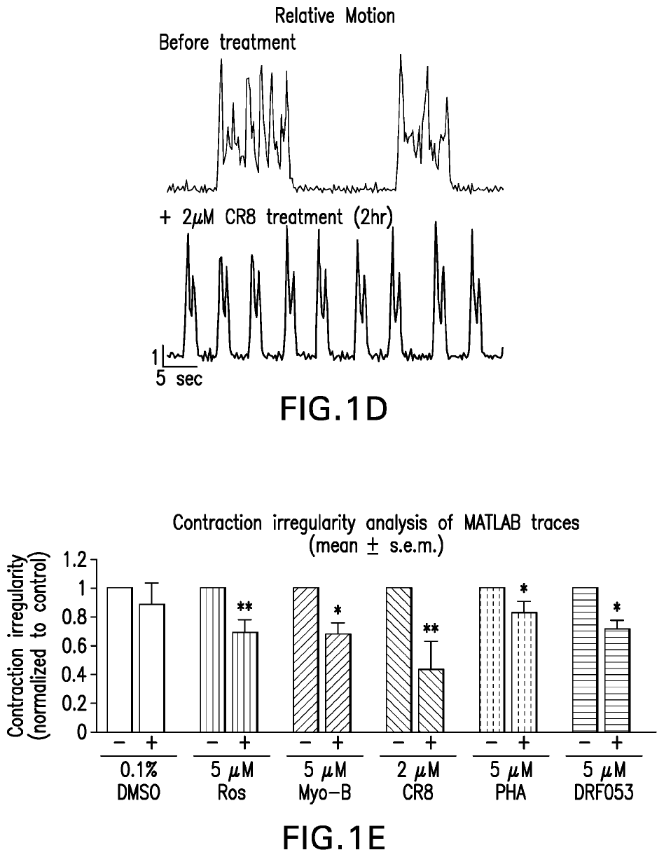

Twenty Roscovitine analogs and four CDK inhibitors with different specificity against CDKs were tested using a contraction assay with Matlab-based analysis (Huebsch et al., 2015; Yazawa et al., 2011) and calcium imaging (FIG. 1A) with the goal of identifying more potent or less toxic Roscovitine analogs and further exploring the mechanisms underlying the effects of Roscovitine on Timothy syndrome cardiomyocytes.

Two rounds of chemical tests were conducted to test the compounds. The first round of chemical test was conducted using Timothy syndrome cardiomyocyte clusters isolated from the monolayer cardiomyocytes to screen the compounds efficiently and to identify the positive compounds that could increase the spontaneous beating rate and decrease the contraction irregularity of the Timothy syndrome cardiomyocyte clusters (FIG. 1C). A second round of chemical test was conducted using the intact monolayer cardiomyocytes to validate the beneficial effects of the positive compounds on Timothy syndrome cardiomyocytes and eliminate the potential bias that could be caused by mechanical isolation (FIGS. 1B-1E).

From the two rounds of chemical test, we identified two Roscovitine analogs, CR8 and Myoseverin B, and two CDK inhibitors, PHA-793887 and DRF053, that have beneficial effects on Timothy syndrome cardiomyocytes (Bettayeb et al., 2008; Brasca et al., 2010; Meijer et al., 1997; Oumata et al., 2008) (FIGS. 1B-1E). After summarizing the CDK targets of the positive compounds, a common element among the positive compounds was identified, which is that four of the five positive compounds have been reported to inhibit CDK5 (Bettayeb et al., 2008; Brasca et al., 2010; Meijer et al., 1997; Oumata et al., 2008) (FIG. 1B), suggesting that CDK5 is likely to be one of the key molecular mechanisms underlying Timothy syndrome.

FIG. 1C provides data showing that eighteen Roscovitine analogs did not show positive effect for correcting TS phenotypes, even though some of the compounds were able to increase the spontaneous beating rate of the Timothy syndrome cardiomyocyte clusters.

2. The Effects of CDK5 Inhibition on Timothy Syndrome Cardiomyocytes

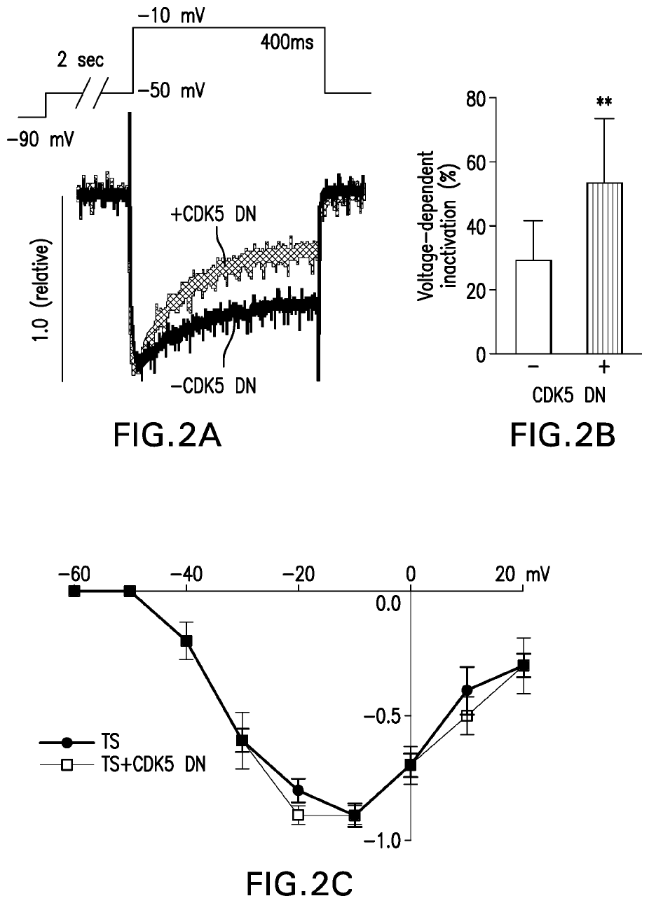

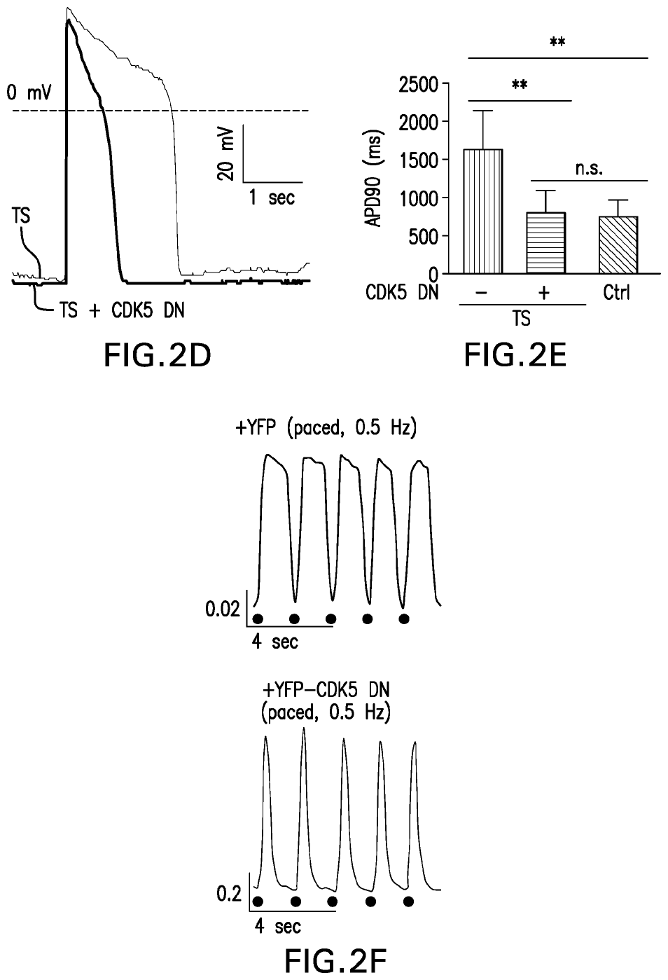

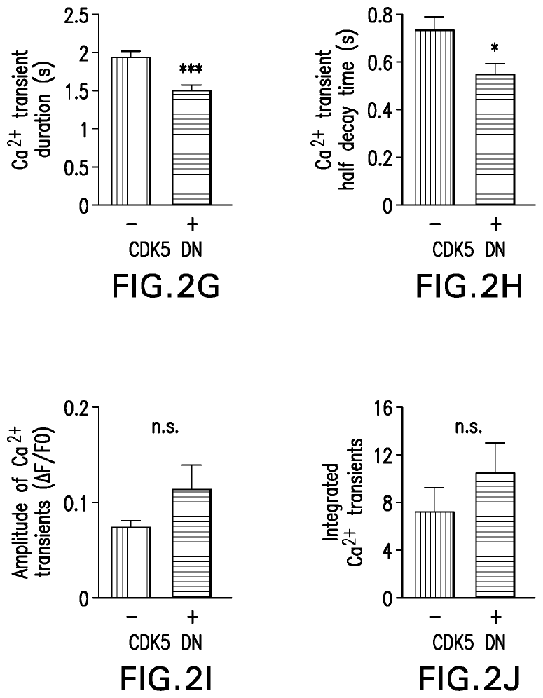

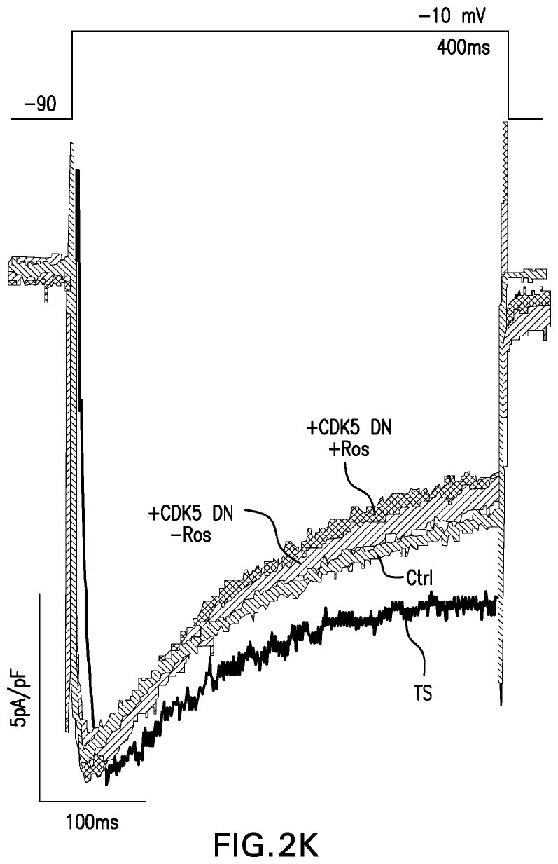

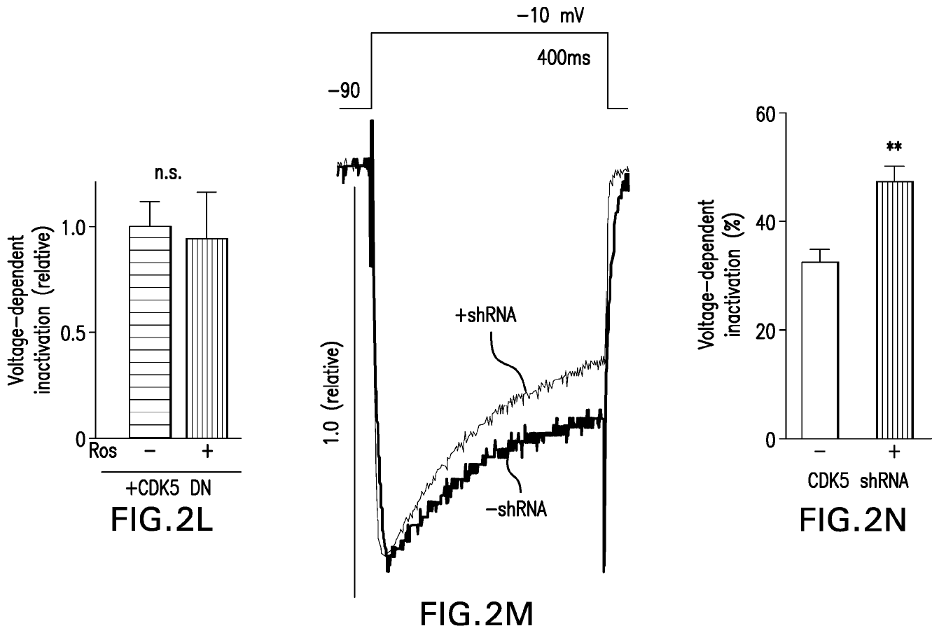

To examine whether CDK5 inhibition is beneficial for TS cardiomyocytes, we first constructed a lentivirus containing the dominant negative (DN) mutant of CDK5. We used patch-clamp recordings and calcium imaging to assess the physiological properties of the TS cardiomyocytes infected with the CDK5 DN lentivirus. The phenotypes of TS cardiomyocytes include a delayed voltage-dependent inactivation of Cav1.2 channels, abnormal action potentials and abnormal calcium transients. The TS cardiomyocytes with CDK5 DN expression demonstrated a significantly enhanced voltage-dependent inactivation of Cav1.2 channels compared with the cardiomyocytes without CDK5 DN expression (FIGS. 2A-2C). Moreover, the expression of CDK5 DN significantly shortened the paced action potential duration and rescued the abnormal action potentials in TS cardiomyocytes (FIGS. 2D, 2E and Table 2). In addition, we examined the effects of CDK5 DN expression on the calcium currents in TS cardiomyocytes. The results suggest that TS cardiomyocytes demonstrated more late calcium currents compared with control cardiomyocytes, and the expression of CDK5 DN significantly reduced the late calcium currents in TS cardiomyocytes. Finally, CDK5 DN expression alleviated the abnormal calcium transients, and significantly reduced the calcium transient duration and half decay time in the paced TS cardiomyocytes (FIGS. 2F-2J). Overall, the results indicated that CDK5 DN expression could alleviate all the previously-reported phenotypes in TS cardiomyocytes.

Next, we examined the effect of Roscovitine on the TS cardiomyocytes infected with the CDK5 DN lentivirus, to investigate whether CDK5 inhibition partially accounts for the therapeutic effects of Roscovitine on TS cardiomyocytes. The results showed that Roscovitine did not further enhance the voltage-dependent inactivation of Cav1.2 in TS cardiomyocytes with CDK5 DN expression, indicating that CDK5 DN expression is sufficient to rescue the delayed voltage-dependent inactivation of Cav1.2 in TS cardiomyocytes (FIGS. 2K and 2L).

To validate our findings using another approach, we designed short hairpin RNA (shRNA) lentiviral constructs that target CDK5 and confirmed the knockdown efficiency of the constructs. We then infected TS cardiomyocytes with the CDK5 shRNA lentivirus and examined the effects of CDK5 shRNA expression on the reported phenotypes in TS cardiomyocytes. CDK5 shRNA expression significantly enhanced the voltage-dependent inactivation of Cav1.2 in TS cardiomyocytes (FIGS. 2M and 2N). In addition, CDK5 shRNA expression alleviated the abnormal spontaneous calcium transients in TS cardiomyocytes. The effects of CDK5 shRNA expression on TS cardiomyocytes were thus similar to the effects of CDK5 DN expression on TS cardiomyocytes, indicating that CDK5 inhibition is beneficial for TS cardiomyocytes.

### Mechanism Underlying the Positive Effects of CDK5-Specific Inhibition on Timothy Syndrome Cardiomyocytes

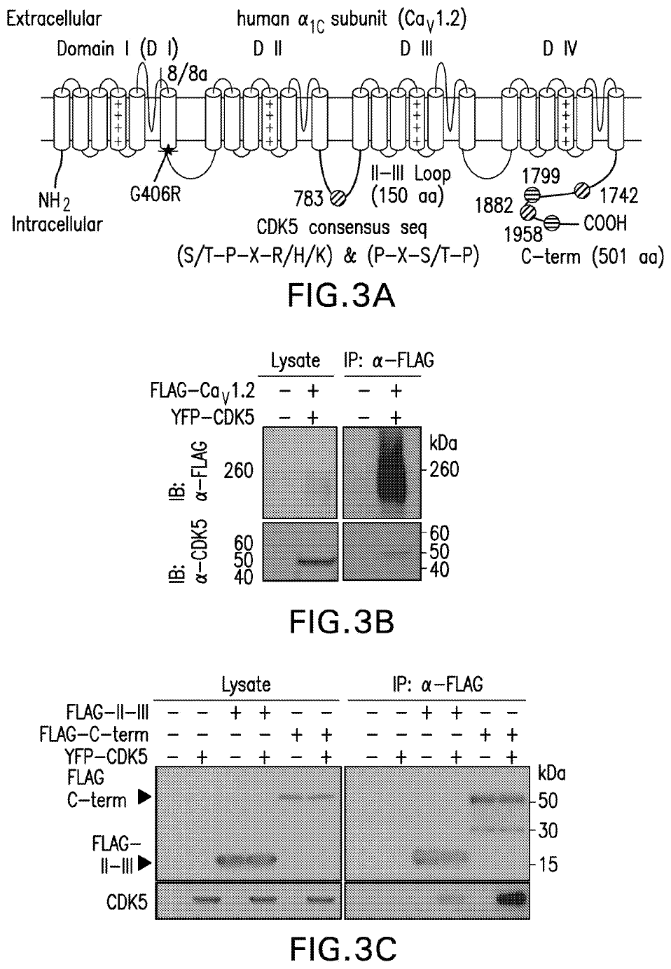

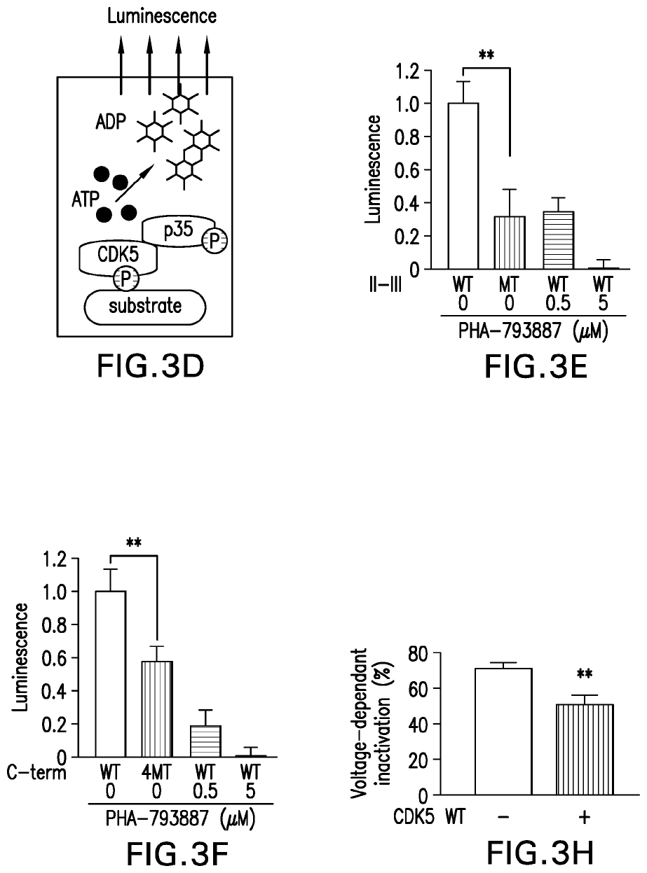

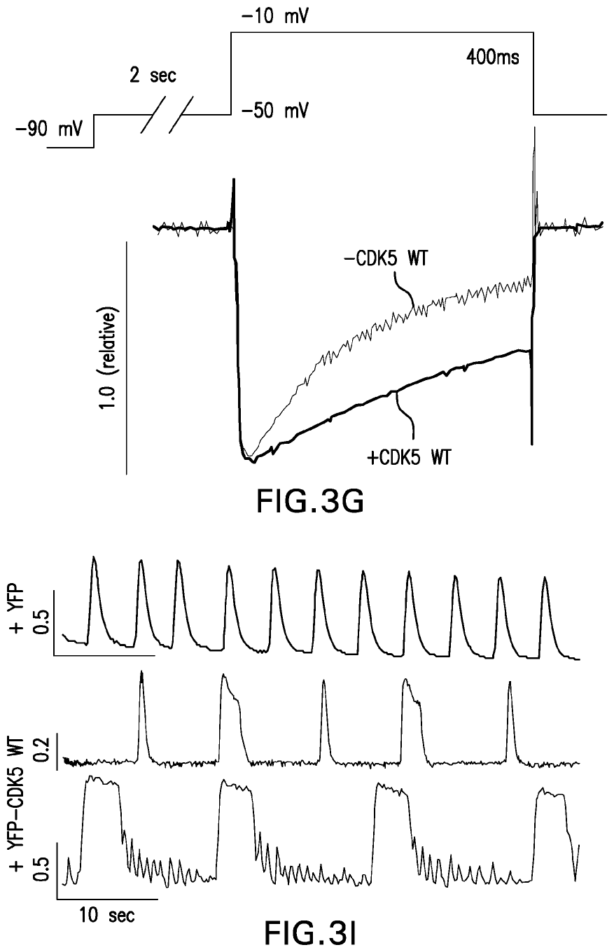

The positive effects of CDK5-specific inhibition on Timothy syndrome cardiomyocytes prompted further investigation of the underlying mechanisms. CDK5 has been reported to phosphorylate serine or threonine in two consensus sequences, S/T-P-X-R/H/K and P-X-S/T-P (X is any amino acid) (Dhariwala and Rajadhyaksha, 2008; Plattner et al., 2014). The sequences of Cav1.2 channels were examined and five consensus sequences located at the II-III loop and the Carboxyl-terminus (C-term) were identified, which are conserved in both human and rodent (FIG. 3A). Plasmids containing FLAG-tagged full length Cav1.2 and YFP-tagged CDK5 were generated for a co-immunoprecipitation (co-IP) assay. The co-IP result demonstrated a binding of CDK5 with Cav1.2 (FIG. 3B). Next, FLAG-tagged II-III loop and FLAG-tagged C-term of Cav1.2 constructs were generated to repeat the co-IP assay, and validated the binding of CDK5 with the two fragments (FIG. 3C).

To determine whether CDK5 phosphorylates Cav1.2, an in vitro kinase assay was designed. The wild-type II-III loop or the C-term of Cav1.2 was used as the substrates in this assay. Mutant II-III loop and mutant C-term constructs were generated with substitutions of serine/threonine to glycine or alanine in all CDK5 consensus sequences as negative controls. The phosphorylation of the substrates by CDK5 consumes ATP and produces ADP, which is then converted into luminescence by detection reagents in the assay (FIG. 3D). The luminescence signal was reduced when the CDK5 inhibitor, PHA-793887 was added to the reactions using wild-type II-III loop or C-term as the substrates. Moreover, the luminescence signal was significantly reduced when using mutant II-III loop or C-term as the substrates when compared to using wild-type II-III loop or C-term as the substrates in the kinase reactions. The results indicated the phosphorylation of the II-III loop and the C-term of Cav1.2 by CDK5 in vitro (FIGS. 3E-3F). The remaining signals in the mutant II-III loop and C-term could come from the phosphorylation of p35 by CDK5 (Asada et al., 2012) and/or non-specific phosphorylation of some serine/threonine residues in the mutant II-III loop and C-term. To provide additional support for the in vitro biochemical results, tests were designed to determine whether wild-type CDK5 over-expression alters Cav1.2 channel functions in control cardiomyocytes. The results illustrated that wild-type CDK5 over-expression significantly delayed the voltage-dependent inactivation of Cav1.2 (FIGS. 3G-3H) and induced abnormal calcium transients in control cardiomyocytes (FIG. 3I). Taken together, the results demonstrated that CDK5 potentially affects Cav1.2 functions by direct binding and phosphorylation and that CDK5 over-expression could result in a significantly delay in the voltage-dependent inactivation of Cav1.2 in control cardiomyocytes.

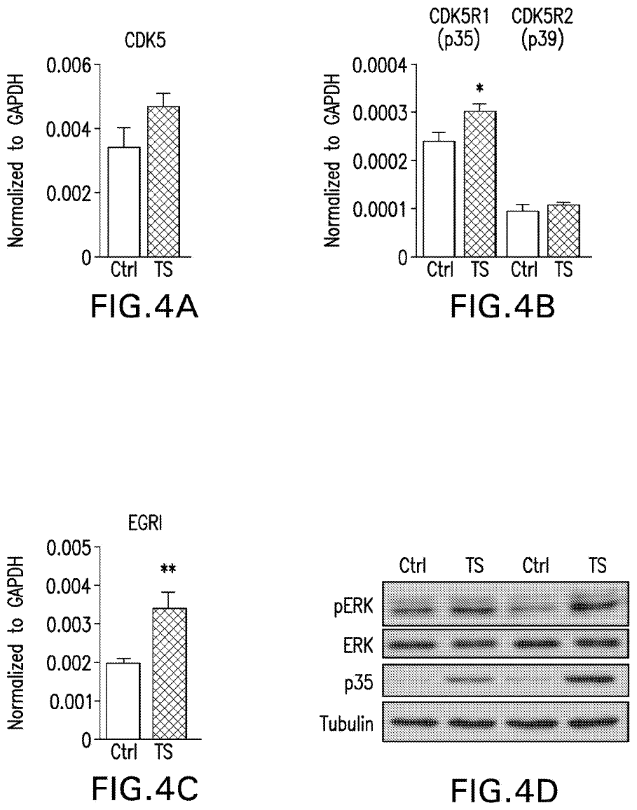

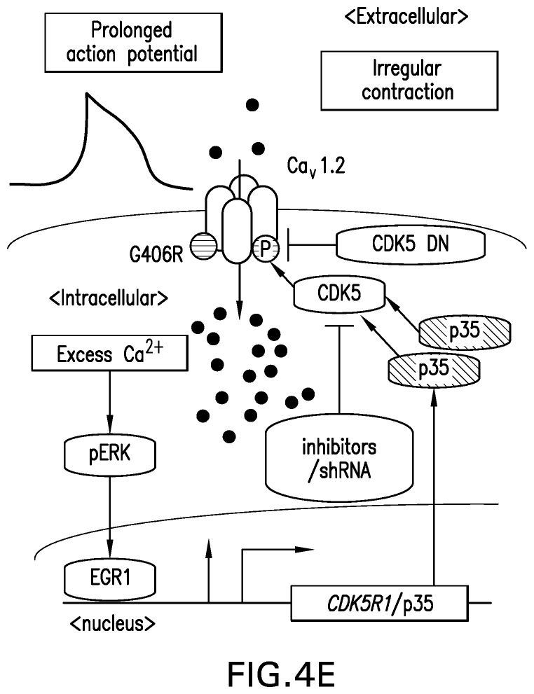

To further explore the signaling pathways underlying the effects of CDK5 inhibition on Timothy syndrome cardiomyocytes, the mRNA expression of CDK5 and its activator p35 (CDK5R1) and p39 (CDK5R2) were measured in control and Timothy syndrome cardiomyocytes. A significant increase in the mRNA expression of p35 was detected in Timothy syndrome cardiomyocytes compared with controls (FIGS. 4A-4B). The expression of EGR1, a transcription factor that regulates p35 transcription (Harada et al., 2001; Shah and Lahiri, 2014), was analyzed and a significant increase in EGR1 mRNA expression was detected in Timothy syndrome cardiomyocytes (FIG. 4C). Furthermore, increased p35 mRNA expression led to an increased p35 protein expression in Timothy syndrome cardiomyocytes (FIG. 4D). The accumulation of p35 protein in Timothy syndrome cardiomyocytes could lead to CDK5 hyper-activation. The protein expression of ERK (Harada et al., 2001; Shah and Lahiri, 2014), the upstream regulator of EGR1 was also analyzed. The results showed that the expression of phosphorylated ERK increased in Timothy syndrome cardiomyocytes, indicating an elevated ERK activity (FIG. 4D). A connection between an increased intracellular calcium concentration and ERK activation in cardiomyocytes (Wheeler-Jones, 2005; Zarain-Herzberg et al., 2011) has previously been established. The present data indicates that a likely scenario that in Timothy syndrome cardiomyocytes is that excessive calcium influx through the mutant Cav1.2 channels causes an increase in ERK activity, resulting in a subsequent induction of EGR1 and an increase in p35 expression. The increased expression of p35 causes CDK5 hyper-activation, which enhances the delayed inactivation of the mutant Cav1.2 channels, leading to more severe phenotypes in the Timothy syndrome cardiomyocytes. Thus, CDK5 inhibition using CDK5 inhibitors, DN or shRNA alleviates the phenotypes in Timothy syndrome cardiomyocytes (FIG. 4E).

### Discussion

Human cardiomyocytes derived from the iPSCs generated from the skin fibroblasts of Timothy syndrome patients were utilized for a phenotypic screen to identify new potential therapeutic compounds based on the chemical structure of Roscovitine and to investigate whether the beneficial effects of Roscovitine on Timothy syndrome cardiomyocytes and whether certain effects are due to inhibitory effects on CDKs. Starting from the screen, a role of CDK5 in the pathogenesis of Timothy syndrome was identified as well as a new mechanism underlying the therapeutic effects of Roscovitine on Timothy syndrome cardiomyocytes. This additional mechanism of action is that Roscovitine exhibits its effects in part by inhibiting CDK5. Roscovitine has been reported to enhance the voltage-dependent inactivation of Cav1.2 with Timothy syndrome mutation in a heterologous over-expression system previously (Yarotskyy and Elmslie, 2007; Yarotskyy et al., 2010; Yarotskyy et al., 2009). Compared with the heterologous over-expression system, the presently tested model system comprising Timothy syndrome cardiomyocytes derived from iPSCs allowed for direct investigation of the effects and mechanisms of Roscovitine in a more physiological-relevant human cardiac environment. This system also allowed for identification of new players, such as CDK5, that is involved in the regulation of Cav1.2 in heart.

The present data demonstrate, for the first time, that CDK5 plays an important role in regulating Cav1.2 functions in cardiomyocytes and the inhibition of CDK5 is beneficial for Timothy syndrome cardiomyocytes. These data provide new insights into the molecular bases of cardiac calcium channel regulation and the development of future therapeutics for Timothy syndrome patients and other patients with related channelopathies.

### Methods

iPSC Maintenance.

iPSCs were cultured with Essential 8 media with 100 unit/ml penicillin and 100 μg/ml streptomycin on plates or dishes (Corning) coated with Geltrex (Life Technologies) following the manufacturer's instruction. The iPSCs were differentiated into cardiomyocytes following a monolayer based protocol that we reported previously (Song et al., 2015).

Plasmid Construction and the Preparation of Lentiviruses.

The CDK5 cDNA was amplified from the cDNA samples of a control iPSC line using Phusion polymerase (Thermo Scientific) and with primer sets that allowed us to add restriction enzyme site NotI and Kozak sequence before the start codon and another site XhoI after the stop codon. The fragment was subcloned into a pcDNA3 vector (Invitrogen) that was digested with NotI and XhoI for the following generation of CDK5 WT and CDK5 dominant negative (DN) lentiviruses. For the generation of CDK5 dominant negative mutant (DN), the QuikChange II XL Site-Directed Mutagenesis Kit (Agilent) was used to generate the mutation leading to the D144N mutation in CDK5 protein. The plasmid containing the CDK5 DN or CDK5 WT was used as the templates to amplify the CDK5 DN or CDK5 WT sequence using Phusion polymerase and the primer sets that allowed us to add restriction enzyme site EcoRI and Kozak sequence before the start codon and another site XhoI after the stop codon. The PCR products were subcloned into lentiviral vector that was prepared from LV-SD-Cre (Addgene, #12105, no longer available currently) digested with EcoRI and XhoI. XL-10 Gold competent cells (Agilent) transformed with the lentiviral LV-SD vectors were inoculated at 24-30° C. The purified LV-SD vectors were transfected together with pCMV-dR8.2 dvpr and pCMV-VSV-G (Addgene #8455 & 8454) into HEK 293T cells for lentiviral production, following a protocol described previously (Song et al., 2015). The shRNA constructs for CDK5 were purchased from GeneCopoeia along with the Lenti-Pac FIV Expression Packaging Kit (FPK-LvTR-40). The knockdown efficiency of the shRNAs was examined and the scrambled shRNA (as a control) lentivirus and CDK5 shRNA lentivirus were prepared in the lentiviral 293Ta packaging cells (Lenti-Pac, # CLv-PK-01) purchased from GeneCopoeia, following the manufacturer's instructions. The shRNA lentiviruses were concentrated 6 folds using the Lenti-X concentrator (Clontech) following the manufacturer's instructions to infect the Timothy syndrome cardiomyocytes. The FLAG-tagged full-length rat CaV1.2 plasmid, the FLAG-II-III loop plasmid and the FLAG-C-terminus plasmid were generated using conventional sub-cloning method using Phusion and PCR primers in pcDNA3 vector as described above. The QuikChange II XL Site-Directed Mutagenesis Kit was used to introduce the mutation(s) to the FLAG-II-III loop plasmid and the FLAG-C-terminus plasmid leading to S783G mutation in the II-III loop amino acid sequence and 51742A/51799A/51882A/T1958A mutations in the C-terminus amino acid sequence.

The Analysis of Cardiomyocyte Contractions for Compound Test.

The working solution of each compound was made by diluting the stock solution in our cardiomyocyte culture media to a final concentration of 5 μM except for (R)-CR8, which was diluted to a final concentration of 1 or 2 μM. The contraction analysis was performed as reported previously (Yazawa et al., 2011). The movies were taken before the treatment, and 24 hours after the treatment of each compound from the Timothy syndrome cardiomyocyte clusters isolated from the monolayer cardiomyocytes for the first round of test. The movies were taken before the treatment, and 2 hours after the treatment of each positive compound on the intact monolayer Timothy syndrome cardiomyocytes for the second round of test. The contraction rate and the irregularity of each sample before and after treatment were compared using paired Student t-test.

Patch-Clamp Electrophysiology.

The Timothy syndrome and control iPSCs were differentiated into cardiomyocytes following a protocol reported previously (Song et al., 2015) and infected with the lentiviruses at day 19-21 or day 25-27 after cardiac differentiation. The cardiomyocytes were dissociated into single cells for whole-cell patch-clamp recordings at day 31. Whole-cell patch-clamp recordings of iPSC-derived cardiomyocytes were conducted using a MultiClap 700B patch-clamp amplifier (Molecular Devices) and an inverted microscope equipped with differential interface optics (Nikon, Ti-U). The glass pipettes were prepared using borosilicate glass (Sutter Instrument, BF150-110-10) using a micropipette puller (Sutter Instrument, Model P-97). Voltage-clamp measurements were conducted using an extracellular solution consisting of 5 mM BaCl₂, 160 mM TEA and 10 mM HEPES (pH7.4 at 25° C.) and a pipette solution of 125 mM CsCl, 0.1 mM CaCl₂, 10 mM EGTA, 1 mM MgCl2, 4 mM MgATP and 10 mM HEPES (pH 7.4 with CsOH at 25° C.). Two pulse protocols were used. One protocol was that cells were held at −90 mV and then depolarized to −10 mV for 400 ms at a rate of 0.1 Hz for the Ba²⁺ current recordings. The other protocol was that cells were held at −90 mV and depolarized to −50 mV for 2 seconds to eliminate the T-type current contamination, and then depolarized to −10 mV for 400 ms at a rate of 0.1 Hz for the Ba²⁺ current recordings to record the L-type current precisely; cells were held at −90 mV, stimulated with a 2-s family of pulses from −90 to +20 for the current-voltage relationship of the Ba²⁺ currents. The recordings were conducted under room temperature. Current-clamp recordings were conducted in normal Tyrode solution containing 140 mM NaCl, 5.4 mM KCl, 1 mM MgCl₂, 10 mM glucose, 1.8 mM CaCl₂ and 10 mM HEPES (pH7.4 with NaOH at 25° C.) using the pipette solution: 120 mM K D-gluconate, 25 mM KCl, 4 mM MgATP, 2 mM NaGTP, 4 mM Na₂-phospho-creatin, 10 mM EGTA, 1 mM CaCl₂ and 10 mM HEPES (pH 7.4 with KCl at 25° C.). The recordings were conducted at 37° C. (R)-Roscovitine stock solution was diluted with the extracellular solution into a working solution of 5 μM and the same concentration of DMSO was used as a control. Current-clamp recordings for paced action potentials were conducted in normal Tyrode solution containing 140 mM NaCl, 5.4 mM KCl, 1 mM MgCl₂, 10 mM glucose, 1.8 mM CaCl₂ and 10 mM HEPES (pH7.4 with NaOH at 25° C.) using the pipette solution: 120 mM K D-gluconate, 25 mM KCl, 4 mM MgATP, 2 mM NaGTP, 4 mM Na₂-phospho-creatin, 10 mM EGTA, 1 mM CaCl₂ and 10 mM HEPES (pH 7.4 with HCl at 25° C.). The recordings were conducted at 37° C. Cardiac action potentials were stimulated (5 ms, 0.3 nA) in current clamp mode at 37° C. (0.2 Hz). First, we paced the patient cardiomyocytes at 0.5 Hz or 1 Hz for action potential recordings and we found that the cardiomyocytes could not respond the pacing frequencies due to the prolonged action potential phenotype (>2 seconds). Therefore, we decided to use lower pacing frequency (0.2 Hz) and examined the effects of CDK5 DN expression on the paced action potentials in Timothy syndrome cardiomyocytes. Recorded action potentials were analyzed using Clampfit 10.4 (Axon Instruments).

Calcium Imaging.

For the paced calcium transient recordings to examine the effects of CDK5 DN expression on the abnormal paced calcium transients in Timothy syndrome cardiomyocytes, the cardiomyocytes were prepared with the same experimental schedule as described in electrophysiology method section. The Nikon automatic microscope (Nikon Eclipse TiE with a motorized stage) connected to sCMOS camera (Andor Zyla sCMOS 4.2 MP) together with a stage top incubator (at 37° C., 5% CO₂ and 20% O₂, controlled by TOKAI HIT Hypoxia gas delivery system) were used for this experiment. Nikon objective lens 40× (Nikon CFI Plan Apo Lambda, NA 0.95) was used for single cell recordings and the normal Tyrode solution with 10% FBS was used as bath solution. A stimulus isolation unit (Warner instruments, SIU-102) and a perfusion insert with electric field stimulation for 35 mm dish (Warner instruments, RC-37FS) were used for electrical pacing. The stimulus isolation unit was set at Bipolar pulse and 4 volts. The pulses were controlled by the Nikon NLS-element software and were given at a frequency of 0.5 Hz with a duration of 2 ms. The parameters (Bipolar pulse, 4 volts, 2 ms, 0.5 Hz) used for the experiments were first optimized using the control cardiomyocytes and control cardiomyocytes responded to the electrical pulses given with this set of parameters. Identical pacing parameters and experimental setting were used for the Timothy syndrome cardiomyocytes with YFP expression and the Timothy syndrome cardiomyocytes with YFP-CDK5 DN expression. The calcium transient duration, amplitude, integrated calcium transients and the calcium transient half (50%) decay time were analyzed.

Co-Immunoprecipitation and Western Blot Analysis.

Co-immunoprecipitation was performed with the Anti-FLAG M2 Affinity Gel (Catalog # A2220, Sigma-Aldrich) and the 293T cell lysates expressing the target proteins. The Tris-HCl based SDS-PAGE gels with 5% stacking gel and 10% separation gel were used for SDS-PAGE. Anti-FLAG antibody (Mouse mAb, Catalog # F3165, Clone # M2, Sigma Aldrich) and Anti-CDK5 antibody (Rabbit mAb, Catalog # ab40773, Clone # EP716Y, Abcam) were used for the immunoblotting. For western blot analysis, the cardiomyocytes were collected at day 26 or day 27 after differentiation and lysed with the cell lysis buffer. The concentration of total proteins in each sample was measured using a standard bicinchoninic acid (BCA) assay kit (Pierce Biotechnology) and 20 μg of proteins from each sample was denatured and loaded to the Tris-HCl based SDS-PAGE gels with 5% stacking gel and 10% separation gel. Anti-ERK1/2 antibody (Mouse mAb, Catalog #9107, Clone #3A7, Cell Signaling), Anti-Phospho-ERK1/2 antibody (Rabbit mAb, Catalog #4370, Clone # D13.14.4E, Cell Signaling), Anti-p35 (Rabbit polyclonal Ab, Catalog # sc-820, Clone # C-19, Santa Cruz) and Anti-beta-Tubulin antibody (Mouse mAb, Catalog # T5201, Clone # TUB 2.1, Sigma Aldrich) were used for immunoblotting.

In Vitro Kinase Assay.

To prepare the substrates, the HEK 293T cells were transfected with the plasmid containing either the FLAG-tagged wild-type II-III loop or mutant II-III loop or wild-type C-terminus or mutant C-terminus using Lipofectamine 2000 (Thermo Fisher Scientific) following the manufacturer's protocol 24 hours after plating. The cells were lysed 48 hours after the transfection with the cell lysis buffer and then were incubated with the Anti-FLAG M2 Affinity Gel for 2 hours at 4° C. After the incubation, the resins were washed and distributed into multiple tubes and each tube contains 10 μl packed resins. For the kinase reactions, the 5× Reaction Buffer A, DTT (0.1M), CDK5/p35 (0.1 μg/μ1), ADP-Glo™ reagent, detection reagent, UltraPure ATP and ADP were purchased from Promega (CDK5/p35 kinase enzyme system, Catalog # V3271, ADP-Glo™ kinase assay, Catalog # V9101, Promega). The final kinase reaction mix contains 10 μl packed resins (substrate), 1× Reaction Buffer A, 50 μM DTT, 50 μM ATP, 0.1 μg CDK5/p35 in distilled water. The stock of PHA-793887 was diluted with DMSO and added to the corresponding samples in PHA-treated groups. The same volume of DMSO was added to the rest of the samples to achieve the same concentration of DMSO in all the reactions. A series of samples for a standard curve were prepared based on the manufacturer's instructions to determine the ATP-ADP conversion from the luminescence signals in every round of experiment. The kinase reaction tubes with the reaction mixes were incubated at 26-27° C. for 60 minutes for the kinase reaction. The ADP-Glo™ reagent was then added to the reactions for an incubation of 40 minutes at 26-27° C. to deplete the ATP in the reactions. Next, the detection reagent was added for an incubation of 45 minutes at 26-27° C. 20 ul of the samples from each tube was then transferred into a 96 well microplate and the luminescence was measured with the GloMax® 96 Microplate Luminometer (Promega).

Statistical Analysis.

The statistics used for every figure have been indicated in the corresponding figure legends. The Student t-test (paired and unpaired) was conducted with the t-test functions in Microsoft Excel software. The Student t-test was two tails. The One-way ANOVA with Bonferroni posthoc analysis was conducted with the Graphpad prism software. All the data meet the assumptions of the statistical tests. All the samples used in this study were biological repeats, not technical repeats. No samples were excluded from the analysis in this study.

Table 1 provides detailed information describing the iPSC lines used for each experiment.

TABLE 1
Methods relating to iPS cell lines
used for the referenced experiments

| | Figure | The information of the cell |
|---|---|---|
| Experiment | number | lines used for the experiment |
| Contraction | FIG. 1D | TS monolayer cardiomyocytes were |
| assay | | differentiated from one iPSC clone(TS1- |
| | | E3-5) from one TS patient (TS1). |
| Voltage-clamp | FIG. 2A-2L | The TS cardiomyocytes were |
| recording | | differentiated from two iPSC clones (TS1- |
| & action | | E3-5 and TS2-E7-1) derived from two |
| potential | | TS patients (TS1 and TS2). The samples |
| recording | | were collected from three rounds of |
| | | differentiation and viral infection. |
| Voltage-clamp | FIG. 3G&3H | The control cardiomyocytes differentiated |
| recording | | from three iPSC clones (IM-E1-5, NH- |
| | | E1-1 and NH-E5-4) derived from two |
| | | different commercially available healthy |
| | | fibroblasts were used. The samples were |
| | | collected from four rounds of |
| | | differentiation and viral infection. |
| Calcium | FIG. 3I | The control cardiomyocyte clusters |
| imaging | | differentiated from two iPSC clones |
| | | (IM-E1-5 and NH-E1-1) derived from |
| | | two different commercially available |
| | | healthy fibroblasts were used. The |
| | | samples were collected from two rounds |
| | | of differentiation and viral infection. |
| Quantitative | FIG. 4A-4C | Some of the control cardiomyocyte |
| PCR | | samples were differentiated from two |
| | | iPSC clones (IM-E1-5 and NH-E1-1) |
| | | derived from two different commercially |
| | | available healthy fibroblasts. The rest |
| | | were differentiated from isogenic control |
| | | iPSC clone 1 and clone 2 that were |
| | | generated from the TS iPSC clones (TS1- |
| | | E3-5 and TS2-E7-1) (See also FIG. S1 for |
| | | isogenic control characterization). TS |
| | | cardiomyocyte samples were from two |
| | | iPSC clones (TS1-E3-5 and TS2-E7-1) |
| | | derived from two TS patients (TS1 and |
| | | TS2). |
| Western blot | FIG. 4D | The experiments were repeated four times |
| analysis | | with different samples to examine p35 |
| | | protein expression. The control cardio- |
| | | myocyte samples were collected from |
| | | two iPSC clones (IM-E1-5 and NH-E1-1) |
| | | derived from two different commercially |
| | | available healthy fibroblasts, and |
| | | isogenic control clone 1 that was |
| | | generated from one of the TS iPSC clones |
| | | (TS1-E3-5) (See also FIG. S1 for |
| | | isogenic control characterization). |
| | | The TS cardiomyocyte samples were |
| | | from two iPSC clones (TS1-E3-5 and |
| | | TS2-E7-1) derived from two TS patients |
| | | (TS1 and TS2). The experiments were |
| | | repeated three times with different |
| | | samples to examine ERK and |
| | | phosphorylated ERK protein expression. |
| | | The control cardiomyocyte samples were |
| | | collected from two iPSC clones (IM-E1-5 |
| | | and NH-E5-4) derived from two different |
| | | commercially available healthy |
| | | fibroblasts, and isogenic control clone |
| | | 4 that was generated from one of the |
| | | TS iPSC clones (TS1-E7-1) (See also FIG. |
| | | S1 for isogenic control characterization). |
| | | The TS cardiomyocyte samples were |
| | | from two iPSC clones (TS1-E3-5 and |
| | | TS2-E7-1) derived from two TS |
| | | patients (TS1 and TS2). Consistent |
| | | results were found in the experiments |
| | | and representative images from one of |
| | | the experiments were shown. |

The detailed generation/characterization of the iPSC clones, the cardiac differentiation method and the use of calcium indicator R-GECO1 for calcium imaging have been reported previously (Song et al., 2015). The names of each iPSC clones described here are the same with our previous publication.

TABLE 2
Raw Data from the paced action potential recordings from single
TS cardiomyocytes with and without CDK5 DN expression.

| Parameter | Group | Mean | s.e.m. | n |
|---|---|---|---|---|
| Resting membrane potential | TS cardiomyocytes | −50.6 | 3.57 | 8 |
| (mV) | without CDK5 DN | | | |
| | TS cardiomyocytes | −53.0 | 4.08 | 10 |
| | with CDK5 DN | | | |
| Peak amplitude | TS cardiomyocytes | 29.3 | 3.5 | 8 |
| (mV) | without CDK5 DN | | | |
| | TS cardiomyocytes | 27.3 | 3.4 | 10 |
| | with CDK5 DN | | | |
| APD90^{a} | TS cardiomyocytes | 1,624.4 | 182.0 | 8 |
| (ms) | without CDK5 DN | | | |
| | TS cardiomyocytes | 801.3 | 93.7 | 10 |
| | with CDK5 DN | | | |
| Time to peak | TS cardiomyocytes | 92.4 | 30.4 | 8 |
| (ms) | without CDK5 DN | | | |
| | TS cardiomyocytes | 40.1 | 11.4 | 10 |
| | with CDK5 DN | | | |
| Maximal upstroke | TS cardiomyocytes | 42.4 | 1.84 | 8 |
| velocity (mV/ms) | without CDK5 DN | | | |
| | TS cardiomyocytes | 40.0 | 2.25 | 10 |
| | with CDK5 DN | | | |

^{a}APD90: Action Potential Duration at 90% of repolarization.

### REFERENCES

Huikuri H V, et al. (2001). Sudden death due to cardiac arrhythmias N Engl J Med. 345(20):1473-82.
Crotti L, et al. (2013). Calmodulin mutations associated with recurrent cardiac arrest in infants. Circulation. 127(9):1009-17.
Mahida S, et al. (2013). Genetics of congenital and drug-induced long QT syndromes: current evidence and future research perspectives. Journal of interventional cardiac electrophysiology: an international journal of arrhythmias and pacing. 37(1):9-19.
Venetucci L, et al. (2012),Inherited calcium channelopathies in the pathophysiology of arrhythmias. Nature reviews Cardiology. 9(10):561-75.
Paakkari I. (2002). Cardiotoxicity of new antihistamines and cisapride. Toxicol Lett. 127(1-3):279-84. Asada, A., Saito, T., and Hisanaga, S. (2012). Phosphorylation of p35 and p39 by Cdk5 determines the subcellular location of the holokinase in a phosphorylation-site-specific manner. J Cell Sci 125, 3421-3429.
Bettayeb, K., Oumata, N., Echalier, A., Ferandin, Y., Endicott, J. A., Galons, H., and Meijer, L. (2008). CR8, a potent and selective, roscovitine-derived inhibitor of cyclin-dependent kinases. Oncogene 27, 5797-5807.
Boczek, N. J., Miller, E. M., Ye, D., Nesterenko, V. V., Tester, D. J., Antzelevitch, C., Czosek,
R. J., Ackerman, M. J., and Ware, S. M. (2015). Novel Timothy syndrome mutation leading to increase in CACNA1C window current. Heart Rhythm 12, 211-219.
Brasca, M. G., Albanese, C., Alzani, R., Amici, R., Avanzi, N., Ballinari, D., Bischoff, J., Borghi, D., Casale, E., Croci, V., et al. (2010). Optimization of 6,6-dimethyl pyrrolo[3,4-c]pyrazoles: Identification of PHA-793887, a potent CDK inhibitor suitable for intravenous dosing. Bioorg Med Chem 18, 1844-1853.
Corona-Rivera, J. R., Barrios-Prieto, E., Nieto-Garcia, R., Bloise, R., Priori, S., Napolitano, C., Bobadilla-Morales, L., Corona-Rivera, A., Zapata-Aldana, E., Pena-Padilla, C., et al. (2015). Unusual retrospective prenatal findings in a male newborn with Timothy syndrome type 1. Eur J Med Genet 58, 332-335.
Dhariwala, F. A., and Rajadhyaksha, M. S. (2008). An unusual member of the Cdk family: Cdk5. Cell Mol Neurobiol 28, 351-369.
Dick, I. E., Joshi-Mukherjee, R., Yang, W., and Yue, D. T. (2016). Arrhythmogenesis in Timothy Syndrome is associated with defects in Ca(2+)-dependent inactivation. Nat Commun 7, 10370.
Flucher, B. E., and Franzini-Armstrong, C. (1996). Formation of junctions involved in excitation-contraction coupling in skeletal and cardiac muscle. Proc Natl Acad Sci USA 93, 8101-8106.
Furusawa, K., Asada, A., Saito, T., and Hisanaga, S. (2014). The effect of Cyclin-dependent kinase 5 on voltage-dependent calcium channels in PC12 cells varies according to channel type and cell differentiation state. J Neurochem 130, 498-506.
Harada, T., Morooka, T., Ogawa, S., and Nishida, E. (2001). ERK induces p35, a neuron-specific activator of Cdk5, through induction of Egrl. Nat Cell Biol 3, 453-459.
Hennessey, J. A., Boczek, N. J., Jiang, Y. H., Miller, J. D., Patrick, W., Pfeiffer, R., Sutphin, B. S., Tester, D. J., Baraj as-Martinez, H., Ackerman, M. J., et al. (2014). A CACNA1C variant associated with reduced voltage-dependent inactivation, increased CaV1.2 channel window current, and arrhythmogenesis. PLoS One 9, e106982.
Huebsch, N., Loskill, P., Mandegar, M. A., Marks, N. C., Sheehan, A. S., Ma, Z., Mathur, A., Nguyen, T. N., Yoo, J. C., Judge, L. M., et al. (2015). Automated Video-Based Analysis of Contractility and Calcium Flux in Human-Induced Pluripotent Stem Cell-Derived Cardiomyocytes Cultured over Different Spatial Scales. Tissue Eng Part C Methods 21, 467-479.
Jacobs, A., Knight, B. P., McDonald, K. T., and Burke, M. C. (2006). Verapamil decreases ventricular tachyarrhythmias in a patient with Timothy syndrome (LQT8). Heart Rhythm 3, 967-970.
Kawaida, M., Abe, T., Nakanishi, T., Miyahara, Y., Yamagishi, H., Sakamoto, M., and Yamada, T. (2016). A case of Timothy syndrome with adrenal medullary dystrophy. Pathol Int 66, 587-592.
Li, B., Tadross, M. R., and Tsien, R. W. (2016). Sequential ionic and conformational signaling by calcium channels drives neuronal gene expression. Science 351, 863-867.
Meijer, L., Borgne, A., Mulner, O., Chong, J. P., Blow, J. J., Inagaki, N., Inagaki, M., Delcros, J. G., and Moulinoux, J. P. (1997). Biochemical and cellular effects of roscovitine, a potent and selective inhibitor of the cyclin-dependent kinases cdc2, cdk2 and cdk5. Eur J Biochem 243, 527-536.
Oumata, N., Bettayeb, K., Ferandin, Y., Demange, L., Lopez-Giral, A., Goddard, M. L., Myrianthopoulos, V., Mikros, E., Flajolet, M., Greengard, P., et al. (2008). Roscovitine-derived, dual-specificity inhibitors of cyclin-dependent kinases and casein kinases 1. J Med Chem 51, 5229-5242.
Papineau, S. D., and Wilson, S. (2014). Dentition abnormalities in a Timothy syndrome patient with a novel genetic mutation: a case report. Pediatr Dent 36, 245-249.
Pasca, S. P., Portmann, T., Voineagu, I., Yazawa, M., Shcheglovitov, A., Pasca, A. M., Cord, B., Palmer, T. D., Chikahisa, S., Nishino, S., et al. (2011). Using iPSC-derived neurons to uncover cellular phenotypes associated with Timothy syndrome. Nat Med 17, 1657-1662.
Philipp, L. R., and Rodriguez, F. H., 3rd (2016). Cardiac arrest refractory to standard intervention in atypical Timothy syndrome (LQT8 type 2). Proc (Bayl Univ Med Cent) 29, 160-162.
Plattner, F., Hernandez, A., Kistler, T. M., Pozo, K., Zhong, P., Yuen, E. Y., Tan, C., Hawasli, A. H., Cooke, S. F., Nishi, A., et al. (2014). Memory enhancement by targeting Cdk5 regulation of NR2B. Neuron 81, 1070-1083.
Seisenberger, C., Specht, V., Welling, A., Platzer, J., Pfeifer, A., Kuhbandner, S., Striessnig, J., Klugbauer, N., Feil, R., and Hofmann, F. (2000). Functional embryonic cardiomyocytes after disruption of the L-type alphalC (Cav1.2) calcium channel gene in the mouse. J Biol Chem 275, 39193-39199.
Shah, D. P., Baez-Escudero, J. L., Weisberg, I. L., Beshai, J. F., and Burke, M. C. (2012).
Ranolazine safely decreases ventricular and atrial fibrillation in Timothy syndrome (LQT8). Pacing Clin Electrophysiol 35, e62-64.
Shah, K., and Lahiri, D. K. (2014). Cdk5 activity in the brain—multiple paths of regulation. J Cell Sci 127, 2391-2400.
Song, L., Awari, D. W., Han, E. Y., Uche-Anya, E., Park, S. H., Yabe, Y. A., Chung, W. K., and Yazawa, M. (2015). Dual optical recordings for action potentials and calcium handling in induced pluripotent stem cell models of cardiac arrhythmias using genetically encoded fluorescent indicators. Stem Cells Transl Med 4, 468-475.
Splawski, I., Timothy, K. W., Sharpe, L. M., Decher, N., Kumar, P., Bloise, R., Napolitano, C., Schwartz, P. J., Joseph, R. M., Condouris, K., et al. (2004). Ca(V)1.2 calcium channel dysfunction causes a multisystem disorder including arrhythmia and autism. Cell 119, 19-31.
Wei, F. Y., Nagashima, K., Ohshima, T., Saheki, Y., Lu, Y. F., Matsushita, M., Yamada, Y., Mikoshiba, K., Seino, Y., Matsui, H., et al. (2005). Cdk5-dependent regulation of glucose-stimulated insulin secretion. Nat Med 11, 1104-1108.
Wheeler-Jones, C. P. (2005). Cell signalling in the cardiovascular system: an overview. Heart 91, 1366-1374.
Yarotskyy, V., and Elmslie, K. S. (2007). Roscovitine, a cyclin-dependent kinase inhibitor, affects several gating mechanisms to inhibit cardiac L-type (Ca(V)1.2) calcium channels. Br J Pharmacol 152, 386-395.
Yarotskyy, V., Gao, G., Du, L., Ganapathi, S. B., Peterson, B. Z., and Elmslie, K. S. (2010). Roscovitine binds to novel L-channel (CaV1.2) sites that separately affect activation and inactivation. J Biol Chem 285, 43-53.
Yarotskyy, V., Gao, G., Peterson, B. Z., and Elmslie, K. S. (2009). The Timothy syndrome mutation of cardiac CaV1.2 (L-type) channels: multiple altered gating mechanisms and pharmacological restoration of inactivation. J Physiol 587, 551-565.
Yazawa, M., Hsueh, B., Jia, X., Pasca, A. M., Bernstein, J. A., Hallmayer, J., and Dolmetsch, R. E. (2011). Using induced pluripotent stem cells to investigate cardiac phenotypes in Timothy syndrome. Nature 471, 230-234.
Zarain-Herzberg, A., Fragoso-Medina, J., and Estrada-Aviles, R. (2011). Calcium-regulated transcriptional pathways in the normal and pathologic heart. IUBMB Life 63, 847-855.

### INCORPORATION BY REFERENCE

All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

The foregoing written specification is considered to be sufficient to enable one skilled in the art to practice the invention. Various modifications of the invention in addition to those shown and described herein will become apparent to those skilled in the art from the foregoing description and fall within the scope of the appended claims. The entire disclosure of each of the patent documents, including certificates of correction, patent application documents, scientific articles, governmental reports, websites, and other references referred to herein is incorporated by reference herein in its entirety for all purposes. In case of a conflict in terminology, the present specification controls.

### EQUIVALENTS

The invention can be embodied in other specific forms without departing from the spirit or essential characteristics thereof. The foregoing embodiments are to be considered in all respects illustrative rather than limiting on the invention described herein. In the various embodiments of the methods and systems of the present invention, where the term comprises is used with respect to the recited steps or components, it is also contemplated that the methods and systems consist essentially of, or consist of, the recited steps or components. Further, it should be understood that the order of steps or order for performing certain actions is immaterial so long as the invention remains operable. Moreover, two or more steps or actions can be conducted simultaneously.

In the specification, the singular forms also include the plural forms, unless the context clearly dictates otherwise. Unless defined otherwise, all technical and scientific terms used herein have the same meaning as commonly understood by one of ordinary skill in the art to which this invention belongs. In the case of conflict, the present specification will control.

All percentages and ratios used herein, unless otherwise indicated, are by weight.

1. A method for inhibiting CDK5 in a subject in need thereof, comprising administering to the subject an effective amount of CR8, Myoseverin B, PHA-793887, DRF053, or any specific chemical inhibitor for CDK5, any combinations thereof, or a pharmaceutically acceptable salt thereof.

2. The method of claim 1, wherein the subject exhibits one or more symptoms associated with Timothy Syndrome (TS) or a related channelopathy.

3. The method of claim 2, wherein one or more symptoms exhibit improvement and comprise any one or combination of improvements selected from the group consisting of increasing the spontaneous beating rate, decreasing the contraction irregularity, enhancing the voltage-dependent inactivation of CaV1.2 channels, rescuing the abnormal action potentials; and alleviating the abnormal calcium transients in affected or diseased cardiomyocytes.

4. The method of claim 1, further comprising increasing sigma-1 receptor activity in a subject in need thereof, and further comprising administering to the subject an effective amount of fluvoxamine or PRE-084, combinations thereof, or a pharmaceutically acceptable salt thereof.

5. A method for treating Timothy Syndrome (TS) or related channelopathy in a subject in need thereof comprising inhibiting CDK5 activity or CDK5 activator p35 in the subject in an amount to alleviate at least one symptom associated with TS or related channelopathy.

6. The method of claim 5, wherein one or more symptoms exhibiting improvement comprise any one or combination of improvements selected from the group consisting of increasing the spontaneous beating rate, decreasing the contraction irregularity, enhancing the voltage-dependent inactivation of CaV1.2 channels, rescuing the abnormal action potentials; and alleviating the abnormal calcium transients in affected or diseased cardiomyocytes.

7. The method of claim 5, wherein inhibiting CDK5 activity comprises administering an effective amount of CR8, Myoseverin B, PHA-793887, DRF053, or any specific chemical inhibitor for CDK5, any combinations thereof, or a pharmaceutically acceptable salt thereof.

8. A method for treating or reducing risk of a cardiac arrhythmia in a subject in need thereof, comprising administering to the subject a therapeutically effective amount of CR8, Myoseverin B, PHA-793887, DRF053, or any specific chemical inhibitor for CDK5, any combinations thereof, or a pharmaceutically acceptable salt thereof.

9. The method of claim 8, wherein the subject exhibits one or more symptoms associated with Timothy syndrome or a related channelopathy.

10. The method of claim 9, wherein one or more symptoms in the subject exhibit improvement and comprise any one or combination of improvements selected from the group consisting of increasing the spontaneous beating rate, decreasing the contraction irregularity, enhancing the voltage-dependent inactivation of CaV1.2 channels, rescuing the abnormal action potentials; and alleviating the abnormal calcium transients in affected or diseased cardiomyocytes.

11. The method of claim 8, further comprising increasing sigma-1 receptor activity in a subject in need thereof, and further comprising administering to the subject an effective amount of fluvoxamine or PRE-084, combinations thereof, or a pharmaceutically acceptable salt thereof.

12. (canceled)

13. The method of claim 5, wherein the inhibiting is by gene therapy or shRNA treatment.

14. The method of claim 5, wherein the inhibitor of CDK5 is selected from the group consisting of proteins, nucleic acids, and combinations thereof.

15. The method of claim 14, wherein the nucleic acid is selected from the group consisting of antisense oligonucleotide, siRNA, shRNA, and combinations thereof.

16. (canceled)
